(19) United States
(12) Patent Application Publication
(10) Pub. No.: US 2025/0382325 A1
(43) Pub. Date: Dec. 18, 2025

(54) DEPROTECTION PROCESSES AND CATION SCAVENGERS FOR USE IN THE SAME

(71) Applicant: EXACTMER LIMITED, Harrogate (GB)

(72) Inventors: Charles Jarrett-Wilkins, Harrogate (GB); Piers Robert James Gaffney, Harrogate (GB)

(21) Appl. No.: 19/151,208

(22) PCT Filed: Feb. 2, 2024
(86) PCT No.: PCT/GB2024/050299

§ 371 Date: Jul. 25, 2025

(30) Foreign Application Priority Data
Feb. 3, 2023 (GB) 2301579.5

(51) Int. Cl. C07K 1/06; C07H 1/00; C07H 21/00; C07K 19/00; C08G 65/00

(52) U.S. Cl. CPC C07K 1/061; C07H 1/00; C07H 21/00; C07K 19/00; C08G 65/00

(57) ABSTRACT

A process for deprotecting an organic compound is described, involving the use of an acid to remove an acid-labile protecting group, and a cation scavenger to react with the protecting group once it has been removed. Also described are cation scavengers suitable for use in the processes described herein, as well as a method for preparing a defined monomer sequence polymer. The cation scavenger has the following Formula I: A-B-C⁻D⁺ wherein A is a group capable of forming a covalent bond with the acid-labile protecting group once removed from the organic compound; B is absent or is a linking moiety; C⁻ is a negatively charged group; and D⁺ is a counter ion.

### INTRODUCTION

The present invention relates to a process for deprotecting an organic compound involving the use of an acid to remove an acid-labile protecting group, and a cation scavenger to react with the protecting group once it has been removed. The invention also relates to cation scavengers suitable for use in the processes described herein, as well as to the use of the deprotection process in a method for preparing a defined monomer sequence polymer.

### BACKGROUND OF THE INVENTION

Organic synthesis involves the construction of molecules with an underlying skeleton of carbon atoms. The techniques of organic synthesis depend upon selectively performing reactions on specific parts of the overall structure, one at a time, so that after a series of such reactions the desired final product is prepared efficiently.

Apart from hydrogen atoms, the underlying carbon skeleton is usually decorated with heteroatoms (R—XH, where X is, e.g., O, N, S or P), in various possible oxidations states and combinations, and the heteroatoms often constitute the most reactive parts of the structure. Therefore, reactive heteroatoms and carbon-based reactive sites must be passivated to prevent their participation in reactions when such reactions are not desirable.

Heteroatoms and reactive carbon sites are frequently passivated by appending relatively inert structures known as protecting groups, R—X—PG. These protecting groups prevent the otherwise reactive sites from inappropriate modification by a reagent being used for another purpose, or interfering in desired reactions elsewhere on the same molecule by both intra- and intermolecular interactions. Temporary protecting groups are removed partway through a synthesis, allowing the reactive site to participate in reactions when desirable, and permanent protecting groups masking a feature wanted in the final product are removed in the final steps of the synthesis.

Protecting groups are widely used in industry, especially in the pharmaceutical industry. They have been used to prepare small molecule drugs, i.e. species that mainly obey Lipinski's rule of five, although if it is at all possible to avoid their use this will be done to increase synthetic efficiency and to reduce costs. However, the industrial use of protecting groups is expected to increase because of growing interest in primary natural product-like drugs, which lie in the complexity range between small molecules and biologics (proteins, mRNA, antibodies, etc.). These drugs are mostly defined monomer sequence polymers, such as oligonucleotides, oligosaccharides and peptides, identical to, or closely derived from, primary natural products. These molecules are highly decorated with large numbers of reactive heteroatoms. Therefore, large numbers of protecting groups are usually employed in their chemical synthesis.

When it is desirable to expose a feature masked by a protecting group, either during a synthesis (temporary protection), or during final global deprotection (permanent protection), it is desirable that the unblocking process is selective and that it goes to completion. In this regard, the deprotection process should use conditions and reagents that are mild and selective enough so that other features (including other protecting groups during removal of temporary protection) remain intact, and that the underlying chemical structure is not damaged. At the same time, the deprotection conditions and reagents should be strong enough that the reaction is complete, or else the synthesis will be inefficient, contaminated by byproducts, and low yielding.

Many reactions, including unblocking of protecting groups, are equilibria and do not proceed to completion without additional driving factors. A common method of forcing deprotection reactions to completion is to transfer the protecting group to an acceptor, scavenger, or trap. Generally speaking, the efficient completion of any deprotection in solution will be favored by the addition of a scavenger to the reaction.

A range of acid labile permanent protecting groups are widely used in peptide synthesis (e.g. tert-butyl (tBu), tert-butyl oxycarbonyl (Boc), mono-methoxytriphenyl (Mmtr), pentamethyl dihydrobenzofuran sulfonyl (Pbf) etc.), which are removed at the end of the synthesis (e.g., with trifluoroacetic acid (TFA)). In most cases, the acid catalysed deprotection proceeds via a cationic protecting group intermediate that is eventually converted to stable protecting groups debris. During global deprotection, a scavenger is typically added to the reaction, which forms an adduct irreversibly, or nearly irreversibly, with the cationic protecting group debris, thereby preventing back-reactions and side-reactions of the debris with the newly exposed reactive sites.

Acid-labile protecting groups are also used in oligonucleotide synthesis. Several approaches are established for the synthesis of oligonucleotides, including the P(III) based phosphoramidite approach, the P(V) phosphotriester approach and the H-phosphonate approach. All three approaches are stepwise methods for building a sequence-defined oligonucleotide. In each case the synthesis cycle is comprised of a coupling step, or steps, followed by the removal of a temporary protecting group. Typically, a mild acid-labile temporary protecting group (most commonly 4,4′-dimethoxytriphenylmethyl, a.k.a. dimethoxytrityl, Dmtr/DMT) is removed from the 5′-terminus in a process termed “detritylation”, prior to chain extension with the next phosphoramidite, phosphodiester, or H-phosphonate building block. During solution phase deprotection of acid labile 5′-O protecting groups, where separation of product and reagent debris in space is impossible, a cation scavenger becomes essential to drive the reaction to completion. Liquid phase oligonucleotide synthesis (LPOS) is well known, although not commercially. In stepwise LPOS, the 5′-O is frequently protected with an acid-labile protecting group. Acidolytic unblocking of 5′-O-(9-phenyxanthen-9-yl) groups, with similar properties to Dmtr, has been facilitated by addition of pyrrole in DCM (Reese et al.). Dmtr can also be trapped by pyrrole (US 2015/0080565) or 5-methoxyindole (US 2018/0282365). The deprotection of Dmtr has also been facilitated by use of a dodecane thiol scavenger (Shi et al.). It is also reported that the efficiency of detritylation during solid phase oligonucleotide synthesis can be increased by the addition of a cation scavenger (U.S. Pat. No. 5,714,597A).

Scavengers are typically added to a deprotection reaction in large excesses due to the fact that the reversible deprotection equilibrium usually favours the starting materials. Accordingly, the bimolecular reaction of between the scavenger and the cleaved protecting group is slow.

Accordingly, there remains a need for improved organic synthesis deprotection strategies.

The present invention was devised with the foregoing in mind.

### SUMMARY OF THE INVENTION

According to a first aspect of the present invention there is provided a process for deprotecting an organic compound, the process comprising the step of contacting a protected organic compound comprising an acid-labile protecting group with:
(i) an acid capable of removing the acid-labile protecting group from the protected organic compound, and
(ii) a cation scavenger having a structure according to formula I:

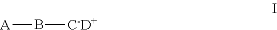

wherein
A is a group capable of forming a covalent bond with the acid-labile protecting group once removed from the organic compound;
B is absent or is a linking moiety;
C⁻ is a negatively charged group; and
D⁺ is a counter ion.

The organic compound is suitably a defined monomer sequence polymer and/or is suitably a polyethylene glycol, oligonucleotide, peptide, peptide nucleic acid or oligosaccharide.

According to a second aspect of the present invention, there is provided a method for the preparation of a defined monomer sequence polymer by sequential coupling of monomeric units, wherein the method comprises one or more deprotection processes of the first aspect.

According to a third aspect of the present invention, there is provided a cation scavenger having a structure according to formula I as defined herein.

### DETAILED DESCRIPTION OF THE INVENTION

Throughout the entirety of the description and claims of this specification, where subject matter is described herein using the term “comprise” (or “comprises” or “comprising”), the same subject matter instead described using the term “consist of” (or “consists of” or “consisting of”) or “consist essentially of” (or “consists essentially of” or “consisting essentially of”) is also contemplated.

Throughout the description and claims of this specification, the singular encompasses the plural unless the context otherwise requires. In particular, where the indefinite article is used, the specification is to be understood as contemplating plurality as well as singularity, unless the context requires otherwise.

Features described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any of the specific embodiments recited herein. The invention extends to any novel one, or any novel combination, of the features disclosed in this specification (including any accompanying claims, abstract and drawings), or to any novel one, or any novel combination, of the steps of any method or process so disclosed.

As discussed hereinbefore, in a first aspect the present invention provides a process for deprotecting an organic compound, the process comprising the step of contacting a protected organic compound comprising an acid-labile protecting group with:
(i) an acid capable of removing the acid-labile protecting group from the protected organic compound, and
(ii) a cation scavenger having a structure according to formula I:

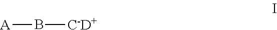

wherein
A is a group capable of forming a covalent bond with the acid-labile protecting group once removed from the organic compound;
B is absent or is a linking moiety;
C⁻ is a negatively charged group; and
D⁺ is a counter ion.

Through rigorous investigations, the inventors have devised an improved process for deprotecting organic compounds bearing acid-labile protecting groups. In particular, the improved process is able to increase the rate of acidic deprotection using a smaller excess of cation scavenger than reported previously. In the process of the invention, a cation scavenger is used, the structure of which has been devised to increase the likelihood of a reaction with cationic protecting group debris that has been acidolytically cleaved from an organic compound. In particular, in the cation scavengers forming part of the invention, reactive group A is tethered to a group, C⁻, the negative charge on which is such that the compound as a whole has an increased affinity for cationic protecting group debris.

Group A is capable of forming a covalent bond with the acid-labile protecting group once it has been removed from the organic compound. It will be understood that removal of the acid-labile protecting group from the organic compound results in the formation of a cationic species, with which group A (or part of group A) is capable of forming a covalent bond. This cationic species may be referred to herein as PG⁺ or cationic protecting group debris.

Without wishing to be bound by theory, acidolytic deprotection of an acid labile protecting group, PG, covalently bonded to a heteroatomic group, X, may be summarised by equations 1 to 4. The overall reaction is represented by Eq. 1. An acid (H-A) is used as a catalyst, often in sub-stoichiometric amounts, to provide a source of protons and the acid's conjugate base, A⁻, in an equilibrium acid dissociation, Eq. 2. A proton then protonates the heteroatom group (X) of the protected starting material, R—XPG, which also dissociates in an equilibrium to liberate the unprotected product, R—XH, and the intermediate protecting group cation, PG⁺, Eq. 3. Equilibria 2 and 3 are driven to the right-hand side by a cation scavenger, Trap-H, that reacts with the protecting group cation, Eq. 4; a proton is liberated in this last step, so that acid is not consumed in the overall reaction, Eq. 1.

$$R - XPG + T rap - H \to R - XH + PG - Trap \qquad Equation 1$$

$$H - A ↔ {H}^{+} + {A}^{-} \qquad Equation 2$$

$$R - XPG + {H}^{+} ↔ {[ R - XHPG ]}^{+} ↔ R - XH + P {G}^{+} \qquad Equation 3$$

$$P {G}^{+} + T rap - H \to PG - Trap + {H}^{+} \qquad Equation 4$$

During deprotection of acid labile protecting groups, a high concentration of scavenger is often necessary to achieve a practical rate of reaction. The scavenger reacts directly with the cationic debris from deprotection, but the concentration of protecting group cation PG⁺ is low because the pseudo-equilibrium between starting material, Eq. 3, usually greatly favours the left-hand side. The rate of the overall reaction can be accelerated by increasing the concentration, or strength of the acid (catalyst), thereby increasing the concentration of cationic intermediate by forcing pseudo-equilibrium Eq. 3 to the right, but this can also increase the rate of side-reactions.

The rate of deprotection is also directly related to the rate of scavenging of the cationic PG⁺, Eq. 4, because this removes cation from the right-hand side of equilibrium Eq. 3. The rate of the overall reaction, Eq. 1, could be increased by substantially raising the concentration of cation scavenger, Trap-H, thereby accelerating its bimolecular reaction with PG⁺, Eq. 4, but adding a large excess of cation scavenger will make the purification of product R—XH more difficult at the end, and may be economically and environmentally costly in terms of scavenger.

The inventors have now devised a means of increasing the rate of successful collisions between the cation PG⁺ and the cation scavenger, Eq. 4, to thereby drive the overall reaction to the right-hand side. This has been achieved by modification of the scavenger's structure in order to introduce an appropriately positioned negative charge that creates an attractive force between the scavenger and PG⁺, bringing them into close proximity, Eq. 5. This attraction, and therefore increased local concentration, will accelerate the scavenging reaction, forming a covalent bond between the scavenger and the protecting group debris.

$$P {G}^{+} + {}^{-} Trap - H ↔ [ {PG}^{+} . {}^{-} Trap - H ] \to PG - Tra {p}^{-} + {H}^{+} \qquad Equation 5$$

As alluded to hereinbefore, those of ordinary skill in the art will be readily familiar with a variety of deprotection strategies for use in organic synthesis. As part of this, those of ordinary skill in the art will be familiar with a variety of acid-labile protecting groups, as well as the types of acids capable of removing these protecting groups, and the types of groups that are able to react with, i.e. scavenge, the resulting cationic protecting group debris. Accordingly, those of ordinary skill in the art will appreciate that the chemistries of the acid, the acid-labile protecting group, and group A are complementary, and will therefore be readily able to select chemically compatible species for each.

In many instances, group A is a nucleophilic group. Group A (or part thereof) may form an adduct with PG⁺ by substitution or reduction. Those of ordinary skill in the art will be familiar with different classes of cation scavengers, in particular reducing, hydride donor-type scavengers (e.g., silanes, such as triethylsilane) and electron-rich, cation acceptor-type scavengers (e.g., 2-mercaptoethanol and electron-rich aromatics prone to electrophilic substitution).

Group A is suitably sulfhydryl, phenolyl, anisolyl, thioanisolyl, electron-rich aryl (e.g., pyrrolyl, indolyl, etc.) or silyl (including alkylsilyl, such as diethylsilyl). Suitably, group A is or comprises a sulfhydryl group. Most suitably, group A is —SH.

Group B may be absent (in which case group A is directly linked to group C⁻) or may be a linking moiety, e.g., an organic linking moiety, and may be aromatic or aliphatic. Suitably, group B is a linking moiety in which the minimum number of bond lengths separating A from C⁻ ranges from 2 to 20. For example, when group B is propylene, the minimum number of bond lengths separating A from C⁻ is 4. Similarly, when group B is phenylene, the minimum number of bond lengths separating A from C⁻ is 3, 4 or 5, depending on whether A and C⁻ are ortho, meta or para to one another. More suitably, group B is a linking moiety in which the minimum number of bond lengths separating A from C⁻ ranges from 3 to 10. Most suitably, group B is a linking moiety in which the minimum number of bond lengths separating A from C⁻ ranges from 3 to 7.

Group B may be a linking moiety comprising at least one of an alkylene group, an alkenylene group, an alkynylene group, an arylene group and a heteroarylene group. Suitably, group B is a linking moiety comprising at least one of an alkylene group and an arylene group. It will be understood that alkylene, alkenylene, and alkynylene groups may be straight or branched (e.g., —CH₂CH₂CH₂CH₂— or —CH₂CH(CH₃)CH₂—). For example, group B may be such that the cation scavenger of formula I takes the form A-alkylene-C⁻D⁺. Alternatively, group B may be such that the cation scavenger of formula I takes the form A-alkylene-arylene-C⁻D⁺. Alternatively, group B may be such that the cation scavenger of formula I takes the form A-arylene-alkylene-C⁻D⁺. Alternatively, group B may be such that the cation scavenger of formula I takes the form A-alkylene-arylene-alkylene-C⁻D⁺. Group B is suitably an alkylene linking moiety or an arylene linking moiety. In the context of group B, arylene is most suitably phenylene (e.g., para-phenylene). More suitably, group B is (2-5C)alkylene or phenylene.

Group B is most suitably ethylene, propylene or phenylene.

The negative charge on group C⁻ serves to increase the likelihood of a reaction between the cation scavenger and the cationic protecting group debris. Group C⁻ may carry a permanent or temporary negative charge. It will be understood that group C⁻ is at least partially negatively charged in the presence of the acid used to remove the acid-labile protecting group.

Group C⁻ may be a conjugate base. It will be understood that the conjugate base must be negatively charged in the conditions under which the process of the invention is performed. Suitably, group C⁻ is a conjugate base of an acid, said acid having a pKa that is more negative than the pKa of the acid capable of removing the acid-labile protecting group from the protected organic compound. This ensures that, in the solvent used for carrying out the process of the invention, group C⁻ remains in its negatively charged conjugate base form. On this basis, those of ordinary skill in the art will, based on their knowledge of acid strengths, be readily able to select an appropriate group C⁻. Indeed, those of ordinary skill in the art will be aware of which acidic groups will, under the conditions used for carrying out the process of the invention, exist as their conjugate base. For example, when the acid used to remove the acid-labile protecting group is trichloroacetic acid, those of ordinary skill in the art will appreciate that simple carboxylic acids are likely to exist in their protonated form, whereas electron-withdrawn carboxylic acids (e.g., fluorinated carboxylic acids) will carry the necessary negative charge. Therefore, group C⁻ may, for example, be the conjugate base of an acid selected from the group consisting of a phenolic acid, a carboxylic acid, a sulfonic acid or a phosphonic acid, said acids having a pKa that is more negative than the pKa of the acid capable of removing the acid-labile protecting group from the protected organic compound. Accordingly, group C⁻ may be a phenolate, a carboxylate (e.g., a fluorinated carboxylate), a sulfonate (including thiosulfonate) or a phosphonate, said groups being at least partially negatively charged in the presence of the acid used to remove the acid-labile protecting group. Purely for illustrative purposes, group C⁻ may, for example, be picrate (i.e., the conjugate base of picric acid), tosylate (i.e., the conjugate base of toluenesulfonic acid) or trichloroacetate (i.e., the conjugate base of trichloroacetic acid).

Group C⁻ is suitably a conjugate base of an acid (e.g., a phenolic acid, a carboxylic acid, a sulfonic acid or a phosphonic acid), said acid having a pKa in acetonitrile that is lower than 12.65. This ensures that if, for example, the process of the invention is carried out in acetonitrile using trifluoroacetic acid as the acid capable of removing the acid-labile protecting group (as is often the case in processes for synthesising oligonucleotides), group C⁻ remains in its negatively charged form. As described hereinbefore, acids such as picric acid (pKa in acetonitrile of ˜11), toluenesulfonic acid (pKa in acetonitrile of ˜8.45) and trichloroacetic acid (pKa in acetonitrile of ˜10.75) will exist as their conjugate base when the process is conducted in acetonitrile using trifluoroacetic acid to remove the protecting group from the protected organic compound.

Group C⁻ is most suitably a sulfonate. In many embodiments, group C⁻ has the structure:

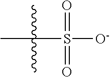

wherein [symbol] denotes the point of attachment to group B (or group A, in situations where group B is absent).

It will be understood that the nature of counter ion D⁺ is such that the cation scavenger is soluble in the solvent used for performing the process of the invention. Accordingly, counter ion D⁺ is suitably an organically-soluble cation. Those of skill in the art will be readily able to select an appropriate counter ion D⁺ based on the nature of groups A, B and C⁻, as well as the solvent in which the process is performed. It will be understood that a proton is not a suitable counter ion D⁺ (e.g., the group C⁻D⁺ is not hydroxy).

Counter ion D⁺ is suitably an alkylammonium ion, an arylammonium ion, an N,N-alkylimidazolium ion, an N-alkylpyridinium ion, an alkylphosphonium ion, an arylphosphonium ion, an alkylarsonium ion or an arylarsonium ion. More suitably, counter ion D⁺ is an alkylammonium ion, such as a (2-4C)alkylammonium ion. The alkylammonium ion may be a tertiary alkylammonium ion or a quaternary alkylammonium ion.

Counter ion D⁺ may alternatively be a soft metal ion, e.g., Cs⁺.

Counter ion D⁺ is most suitably Et₃NH⁺, Et₄N⁺ or Bu₄N⁺.

The cation scavenger may have a structure according to formula Ia:

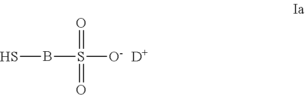

wherein B and D⁺ have any of the definitions outlined hereinbefore.

For cation scavengers of formula Ia, B may be a linking moiety in which the minimum number of bond lengths separating A from C⁻ ranges from 3 to 10. For example, B may be composed of at least one of an alkylene group, an alkenylene group, an alkynylene group, an arylene group and a heteroarylene group. Suitably, group B is a linking moiety composed of at least one of an alkylene group and an arylene group.

For cation scavengers of formula Ia, counter ion D⁺ is suitably an alkylammonium ion, an arylammonium ion, an N,N-alkylimidazolium ion, an N-alkylpyridinium ion, an alkylphosphonium ion, an arylphosphonium ion, an alkylarsonium ion or an arylarsonium ion.

For cation scavengers of formula Ia, counter ion D⁺ is suitably a tertiary alkylammonium ion or a quaternary alkylammonium ion.

The cation scavenger may have a structure according to formula Ib:

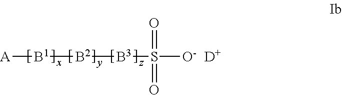

wherein
A and D⁺ have any of those definitions outlined hereinbefore;
B¹ is —CH₂—;
B² is arylene;
B³ is —CH₂—;
x is 0-4;
y is 0 or 1; and
z is 0-4;
with proviso that at least one of x, y and z is not 0.

B² is suitably phenylene (e.g., para-phenylene).

The sum of x and z is suitably ≤5.

When y is 0, x and z are suitably not 0.

When y is 1, x and z are suitably 0-2.

In particular embodiments (i) x is 2-4, and y and z are 0, or (ii) y is 1 and x and z are 0-1. Most suitably (i) x is 2-3, and y and z are 0, or (ii) y is 1, and x and z are 0.

For cation scavengers of formula Ib, group A suitably comprises a sulfhydryl group. Most suitably, group A is —SH.

For cation scavengers of formula Ib, counter ion D⁺ is suitably a tertiary alkylammonium ion or a quaternary alkylammonium ion.

For cation scavengers of formula Ib, counter ion D⁺ is suitably an alkylammonium ion, such as a (2-4C)alkylammonium ion. Most suitably, counter ion D⁺ is Et₃NH⁺, Et₄N⁺ or Bu₄N⁺.

The cation scavenger may have a structure according to formula Ic:

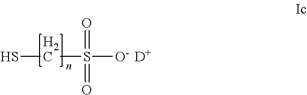

wherein D⁺ has any of the definitions outlined hereinbefore; and
n is 2-8.

n is suitably, 2-6. More suitably, n is 2-5. Most suitably, n is 2-3.

For cation scavengers of formula Ic, counter ion D⁺ is suitably a tertiary alkylammonium ion or a quaternary alkylammonium ion.

For cation scavengers of formula Ic, counter ion D⁺ is suitably an alkylammonium ion, such as a (2-4C)alkylammonium ion. Most suitably, counter ion D⁺ is Et₃NH⁺, Et₄N⁺ or Bu₄N⁺.

The cation scavenger may have a structure according to formula Id:

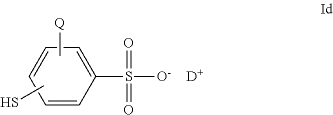

wherein D⁺ has any of the definitions outlined hereinbefore and Q is hydrogen or a substituent having a mass of <100 g mol⁻¹.

Q is suitably hydrogen or a substituent having a mass of <60 g mol⁻¹. Those of ordinary skill in the art will recognise that a variety of substituents can be tolerated by the phenylene group without compromising the efficacy of the A and C⁻ groups (—SH and —SO₃—). Suitably, Q is hydrogen.

For cation scavengers of formula Id, the —SH group is suitably meta or para to the —SO₃⁻ group.

For cation scavengers of formula Id, counter ion D⁺ is suitably a tertiary alkylammonium ion or a quaternary alkylammonium ion.

For cation scavengers of formula Id, counter ion D⁺ is suitably an alkylammonium ion, such as a (2-4C)alkylammonium ion. Most suitably, counter ion D⁺ is Et₃NH⁺, Et₄N⁺ or Bu₄N⁺.

The cation scavenger may have a structure according to any one of formulae Ie, If or Ig:

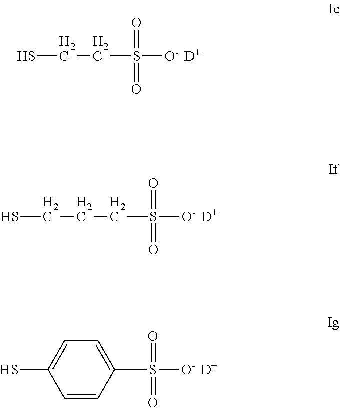

wherein D⁺ has any of the definitions outlined hereinbefore.

For cation scavengers of formula Ie, If, or Ig, counter ion D⁺ is suitably an alkylammonium ion, such as a (2-4C)alkylammonium ion. Most suitably, counter ion D⁺ is Et₃NH⁺, Et₄N⁺ or Bu₄N⁺.

As discussed hereinbefore, the skilled person will be able to select an appropriate acid for removing a given protecting group. The acid capable of removing the acid-labile protecting group from the protected organic compound may, for example, be a haloacetic acid, toluene sulfonic acid, hydrochloric acid, sulfuric acid, phosphoric acid or polyphosphoric acid. Suitably, the acid capable of removing the acid-labile protecting group from the protected organic compound is trifluoroacetic acid, dichloroacetic acid or trichloroacetic acid. Most suitably, the acid capable of removing the acid-labile protecting group from the protected organic compound is trifluoroacetic acid or dichloroacetic acid. Trifluoroacetic acid (e.g., in acetonitrile) is particularly suitable where the organic compound is an oligonucleotide, in which case the protecting group may be DMT. Dichloroacetic acid (e.g., in dichloromethane) is particularly suitable where the organic compound is a defined monomer sequence polymer (e.g., a defined monomer sequence polymer having a polyethylene glycol backbone), in which case the protecting group may be DMT.

The solvent(s) used in the process of the invention will depend on the nature of the organic compound to be deprotected. Those of ordinary skill in the art will be able to select appropriate solvents for carrying out the deprotection of a given organic compound. The step of contacting the protected organic compound with the acid and the cation scavenger may, for example, be conducted in at least one chlorinated hydrocarbon solvent or a polar aprotic solvent. Suitably, the step of contacting the protected organic compound with the acid and the cation scavenger is conducted in dichloromethane, acetonitrile, sulfolane, dimethylformamide, dimethylacetamide, dimethyl sulfoxide, or a mixture of two or more thereof. Alternatively, the step of contacting the protected organic compound with the acid and the cation scavenger may, for example, be conducted in at least one of ethyl acetate, tetrahydrofuran and toluene. Most suitably, the step of contacting the protected organic compound with the acid and the cation scavenger is conducted in dichloromethane or acetonitrile. Acetonitrile may be particularly suitable where the organic compound is an oligonucleotide. Dichloromethane may be particularly suitable where the organic compound is a peptide or a defined monomer sequence polymer having, e.g., a polyethylene glycol backbone.

The organic compound may take a variety of forms. For example, the organic compound may be a relatively small molecule (e.g., a compound having a molecular mass of <500 g mol⁻¹), such as a nucleotide, saccharide or amino acid. Alternatively, the organic compound may be a relatively larger molecule (e.g., a compound having a molecular mass of >500 g mol⁻¹), such as a polymer or an oligomer. In some instances, the polymer or oligomer may be an oligonucleotide, a peptide, a peptide nucleic acid or an oligosaccharide.

The organic compound is suitably a defined monomer sequence polymer. The term “defined monomer sequence polymer” is used in the art to describe a polymer comprising at least two monomers in which at least two of the monomers are distinct from each other, and in which the monomers are present in the same order in the polymer chain for all molecules of the polymer. Defined monomer sequence polymers include peptides, oligonucleotides and oligosaccharides, as well as chemically modified analogues thereof, all of which are biologically important polymeric or oligomeric molecules composed of repeating monomeric units. In the case of peptides the repeating monomeric units are amino acids or their derivatives, while in the case of oligonucleotides the repeating monomeric units are nucleotides or their derivatives, while in the case of oligosaccharides the repeating monomeric units are sugar units or their derivatives.

In many instances, the organic compound is an oligonucleotide, which may be a defined monomer sequence polymer. The oligonucleotide may have at least one backbone modification, and/or at least one sugar modification and/or at least one base modification compared to an RNA or DNA-based oligonucleotide.

The oligonucleotide may contain at least 1 modified nucleotide residue. The modification may be at the 2′ position of the sugar moiety. Sugar modifications in oligonucleotides described herein may include a modified version of the ribosyl moiety, such as 2′-O-modified RNA such as 2′-O-alkyl or 2′-O(substituted)alkyl e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-O-propargyl, 2′-O-allyl, 2′-O-(3-amino)propyl, 2′-O-(3-(dimethylamino)propyl), 2′-O-(2-amino)ethyl, 2′-O-(2-(dimethylamino)ethyl); 2′-deoxy (DNA); 2′-O(haloalkoxy)methyl (Arai K. et al. Bioorg. Med. Chem. 2011, 21, 6285) e.g. 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl](MOCE), 2′-O-[2-(N-methylcarbamoyl)ethyl](MCE), 2′-O-[2-(N,N-dimethylcarbamoyl)ethyl](DCME); 2′-halo e.g. 2′-F, FANA (2′-F arabinosyl nucleic acid); carbasugar and azasuar modifications; 3′-O-alkyl e.g. 3′-O-methyl, 3′-O-butyryl, 3′-O-propargyl; and their derivatives.

Sugar modifications may be selected from the group consisting of 2′-fluoro (2′-F), 2′-O-methyl (2′-OMe), 2′-O-methoxyethyl (2′-MOE), and 2′-amino. Alternatively, the modification may be 2′-O-MOE. Other sugar modifications include “bridged” or “bicylic” nucleic acid (BNA), e.g. locked nucleic acid (LNA), xylo-LNA, α-L-LNA, β-D-LNA, cEt (2′-O,4′-C constrained ethyl) LNA, cMOEt (2′-O,4′-C constrained methoxyethyl) LNA, ethylene-bridged nucleic acid (ENA), tricyclo DNA; unlocked nucleic acid (UNA); cyclohexenyl nucleic acid (CeNA), altritol nucleic acid (ANA), hexitol nucleic acid (HNA), fluorinated HNA (F-HNA), pyranosyl-RNA (p-RNA), 3′-deoxypyranosyl-DNA (p-DNA); morpholino (as e.g. in PMO, PPMO, PMOPIus, PMO-X); and their derivatives.

Oligonucleotides used in the process of the invention may include other modifications, such as peptide-base nucleic acid (PNA), boron modified PNA, pyrrolidine-based oxy-peptide nucleic acid (POPNA), glycol- or glycerol-based nucleic acid (GNA), threose-based nucleic acid (TNA), acyclic threoninol-based nucleic acid (aTNA), oligonucleotides with integrated bases and backbones (ONIBs), pyrrolidine-amide oligonucleotides (POMs); and their derivatives.

The modified oligonucleotide may comprise a phosphorodiamidate morpholino oligomer (PMO), a locked nucleic acid (LNA), a peptide nucleic acid (PNA), a bridged nucleic acid (BNA) such as (5)-cEt-BNA, or a SPIEGELMER.

Modifications may also be present in the nucleobase. Base modifications include modified versions of the natural purine and pyrimidine bases (e.g. adenine, uracil, guanine, cytosine, and thymine), such as inosine, hypoxanthine, orotic acid, agmatidine, lysidine, 2-thiopyrimidine (e.g. 2-thiouracil, 2-thiothymine), G-clamp and its derivatives, 5-substituted pyrimidine (e.g. 5-methylcytosine, 5-methyluracil, 5-halouracil, 5-propynyluracil, 5-propynylcytosine, 5-aminomethyl uracil, 5-hydroxymethyl uracil, 5-aminomethylcytosine, 5-hydroxymethylcytosine, Super 5 T), 2,6-diaminopurine, 7-deazaguanine, 7-deazaadenine, 7-aza-2, 6-diaminopurine, 8-aza-7-deazaguanine, 8-aza-7-deazaadenine, 8-aza-7-deaza-2, 6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), or derivatives thereof; and degenerate or universal bases, like 2,6-difluorotoluene or absent bases like abasic sites (e.g. 1-deoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173. cPent-G, cPent-AP and Pr-AP were shown to reduce immunostimulatory effects when incorporated in siRNA (Peacock et al.).

The nucleobase modification may be selected from the group consisting of 5-methyl pyrimidines, 7-deazaguanosines and abasic nucleotides. Alternatively, the modification may be a 5-methyl cytosine.

Oligonucleotides used in the process of this invention may include a backbone modification, e.g. a modified version of the phosphodiester present in RNA, such as phosphorothioate (PS), phosphorodithioate (PS2), phosphonoacetate (PACE), phosphonoacetamide (PACA), thiophosphonoacetate, thiophosphonoacetamide, phosphorothioate prodrug, H-phosphonate, methylphosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methylboranophosphonothioate, and their derivatives. Another modification includes phosphoramidite, phosphoramidate, N3′˜PS′ phosphoramidate, phosphordiamidate, phosphorothiodiamidate, sulfamate, dimethylenesulfoxide, sulfonate, triazole, oxalyl, carbamate, methyleneimino (MMI), and thioacetamido nucleic acid (TANA); and their derivatives.

Backbone modifications may be selected from the group consisting of: phosphorothioate (PS), phosphoramidate (PA) and phosphorodiamidate. The modified oligonucleotide may be a phosphorodiamidate morpholino oligomer (PMO). A PMO has a backbone of methylenemorpholine rings with phosphorodiamidate linkages.

The oligonucleotide may have a phosphorothioate (PS) backbone.

The oligonucleotide may comprise a combination of two or more modifications as described above. A person skilled in the art will appreciate that there are many synthetic derivatives of oligonucleotides.

The organic compound may alternatively be a gapmer. The 5′ and 3′ wings of the gapmer may comprise or consist of 2′-MOE modified nucleotides. The gap segment of the gapmer may comprise or consist of nucleotides containing hydrogen at the 2′ position of the sugar moiety, i.e., is DNA-like. For example, the 5′ and 3′ wings of the gapmer may consist of 2′-MOE modified nucleotides and the gap segment of the gapmer may consist of nucleotides containing hydrogen at the 2′ position of the sugar moiety (i.e., deoxynucleotides). Alternatively, the 5′ and 3′ wings of the gapmer may consist of 2′-MOE modified nucleotides and the gap segment of the gapmer may consist of nucleotides containing hydrogen at the 2′ position of the sugar moiety (i.e., deoxynucleotides) and the linkages between all of the nucleotides are phosphorothioate linkages.

Defined monomer sequence polymers also include synthetic polymers where at least two distinct monomers are linked in a defined sequence. There is considerable interest in further classes of defined monomer sequence polymers for applications in healthcare (Lutz et al.; Hartmann et al.) and for applications in further industries including flat panel displays where characteristics such as the optical properties of conjugated polymers are important. Acid labile protecting groups have been used in the synthesis of defined monomer sequence polymers with a polyethylene glycol (PEG) backbone (Dong et al.). Therefore, the organic compound may be a defined monomer sequence polymer having a polyethylene glycol backbone.

The acid-labile protecting group will depend on the nature of the organic compound. Those of ordinary skill in the art will be aware of acid-labile protecting groups routinely used with the aforementioned organic compounds. Suitably, the acid-labile protecting group is covalently bonded to a heteroatom (e.g., a N or O atom) of the organic compound. Particular acid-labile protecting groups include dimethoxytrityl (Dmtr or DMT), tert-butyl (tBu), tert-butyl oxycarbonyl (Boc), mono-methoxytriphenyl (Mmtr), triphenylmethyl (Tr), pentamethyl dihydrobenzofuran sulfonyl (Pbf), tetrahydropyranyl (Thp), tetrahydrofuranyl (Thf), para-methoxybenzyl (Pmb) and 2,4-dimethoxybenzyl. Dmtr is a particularly suitable protecting group.

In many instances, the deprotection process of the invention will form part of a larger synthetic procedure. For example, the deprotection process may occur at a midpoint during a larger synthetic procedure, e.g., to expose one or more reactive groups on the organic compound, which is then modified by one or more subsequent reactions. Alternatively, the deprotection process may occur at an endpoint of a larger synthetic procedure, e.g., to expose one or more groups on the organic compound, the reactivity of which would have compromised an earlier step of the procedure.

In the process of the invention, the acid and the cation scavenger may be simultaneously contacted with the protected organic compound. In such instances, the cation scavenger may be able to form an adduct with PG⁺ as soon as it forms (i.e., as soon as the acid has cleaved the acid-labile protecting group from the organic compound).

Alternatively, the acid and the cation scavenger may be sequentially contacted with the protected organic compound. For example, in an initial step, the protected organic compound may be contacted with the acid, after which the resulting PG⁺ is contacted with the cation scavenger.

The process of the invention may further comprise a step of isolating the organic compound, once deprotected. The organic compound, once deprotected, may be isolated by membrane filtration (e.g., membrane diafiltration). In such instances, the porosity of the membrane may be such that the adduct formed by reaction of the cation scavenger and PG⁺ collects in the permeate, whereas the deprotected organic compound collects in the retentate. Suitably, the membrane is an organic solvent-resistant membrane. For example, the organic compound, once deprotected, may be isolated by organic solvent nanofiltration. More suitably, the membrane is insoluble in the solvent(s) used in the step of contacting the protected organic compound with the acid and the cation scavenger. In such cases, the organic compound can be deprotected and isolated in the same solvent(s).

Suitable membranes for use in isolating the organic compound, once deprotected, include polymeric membranes, ceramic membranes, and mixed polymeric/inorganic membranes. Membrane rejection Ri is a common term known by those skilled in the art and is defined as:

$${R}_{i} = ( 1 - \frac{{C}_{Pi}}{{C}_{Ri}} ) \times 100 \% \qquad eq . ( 1 )$$

where C_{P,i}=concentration of species i in the permeate, permeate being the liquid which has passed through the membrane, and C_{R,i}=concentration of species i in the retentate, retentate being the liquid which has not passed through the membrane. It will be appreciated that a membrane is suitable for the invention if

$${R}_{( organic compound )} > {R}_{( cation scavenger - PG adduct )}$$

The membrane may be formed from any polymeric or ceramic material which provides a separating layer capable of preferentially separating the organic compound from the adduct formed by reaction of the cation scavenger and PG⁺. In other words, the membrane will exhibit a rejection for the organic compound that is greater than the rejection for the aforementioned adduct. Suitably, the membrane is formed from or comprises a polymeric material suitable for fabricating microfiltration, ultrafiltration, nanofiltration or reverse osmosis membranes, including polyethylene, polypropylene, polytetrafluoroethylene (PTFE), polyvinylidene difluoride (PVDF), polysulfone, polyethersulfone, polyacrylonitrile, polyamide, polyester, polyimide, polyetherimide, cellulose acetate, polyaniline, polypyrrole, polybenzimidazole (PBI), polyetheretherketone (PEEK) and mixtures thereof. The membranes can be made by any technique known in the art, including sintering, stretching, track etching, template leaching, interfacial polymerisation or phase inversion. Membranes may be composite in nature (e.g., a thin film composite membrane) and/or be crosslinked or treated so as to improve their stability in certain solvents. PCT/GB2007/050218 and PCT/GB2015/050179 describe membranes which may be suitable for use as part of the invention. U.S. Pat. No. 10,913,033 describes a membrane that is particularly suitable for use as part of the invention.

Most suitably, the membrane is a crosslinked polybenzimidazole membrane (e.g., an integrally skinned, asymmetric, crosslinked polybenzimidazole membrane).

As discussed hereinbefore, in a second aspect, the present invention provides a method for the preparation of a defined monomer sequence polymer by sequential coupling of monomeric units, wherein the method comprises one or more deprotection processes according to the first aspect.

The deprotection process of the first aspect is suitably part of a method for preparing a defined monomer sequence polymer (e.g., an oligonucleotide) by stepwise coupling of monomeric units to grow a polymeric chain. In such cases, the protected organic compound is a monomer, oligomer or polymer, deprotection of which exposes a reactive group to which can then be coupled a further monomeric unit. The deprotection process may comprise an isolation step as described herein. The deprotection process (including isolation step) may be performed at several instances throughout the method for preparing a defined monomer sequence polymer. It will be understood that a monomeric unit refers to a polymer building block having a defined structure. The monomeric unit is the minimum repeating unit, factoring in any side chains.

The growing polymeric chain may be attached to a synthesis support during the course of the method for preparing a defined monomer sequence polymer. The synthesis support may serve to retain the growing polymeric chain in solution and/or confer molecular bulk to facilitate isolation by membrane filtration. The synthesis support may, for example, be a branch point molecule, or a polymer, dendrimer, dendron, hyperbranched polymer, or organic/inorganic materials, including nanoparticles, fullerenes and 2-D materials such as graphene and boron nitride. A branch point molecule will be understood to refer to a polyfunctional organic molecular “hub” having a plurality of terminals to which identical growing polymeric chains are attached. Exemplary branch point molecules are outlined in the accompanying examples.

As discussed hereinbefore, in a third aspect the present invention provides a cation scavenger having a structure according to formula I as defined herein.

The following numbered statements 1 to 67 are not claims, but instead describe particular aspects and embodiments of the invention:

1. A process for deprotecting an organic compound, the process comprising the step of contacting a protected organic compound comprising an acid-labile protecting group with:
(i) an acid capable of removing the acid-labile protecting group from the protected organic compound, and
(ii) a cation scavenger having a structure according to formula I:

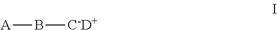

wherein
A is a group capable of forming a covalent bond with the acid-labile protecting group once removed from the organic compound;
B is absent or is a linking moiety;
C⁻ is a negatively charged group; and
D⁺ is a counter ion.
   2. The process of statement 1, wherein A forms a covalent bond with the acid-labile protecting group, once removed from the organic compound, by substitution or reduction.
   3. The process of statement 1 or 2, wherein A is a nucleophilic group.
   4. The process of statement 1, 2 or 3, wherein A is a sulfhydryl, phenolyl, anisolyl, thioanisolyl, electron-rich aryl or silyl.
   5. The process of any one of the preceding statements, wherein A is sulfhydryl.
   6. The process of any one of the preceding statements, wherein B is a linking moiety separating A from C⁻ by a distance of 2-20 bond lengths.
   7. The process of any one of the preceding statements, wherein B is a linking moiety separating A from C⁻ by a distance of 3-10 bond lengths.
   8. The process of any one of the preceding statements, wherein B is a linking moiety separating A from C⁻ by a distance of 3-7 bond lengths.
   9. The process of any one of the preceding statements, wherein B is an organic linking moiety.
   10. The process of any one of the preceding statements, wherein B is an aliphatic or aromatic linking moiety.
   11. The process of any one of the preceding statements, wherein B is a linking moiety formed from at least one of an alkylene group, an alkenylene group, an alkynylene group, an arylene group and a heteroarylene group.
   12. The process of any one of the preceding statements, wherein B is a linking moiety formed from at least one of an alkylene group and an arylene group (e.g., phenylene).
   13. The process of any one of the preceding statements, wherein B is a linking moiety of the form -alkylene-, -arylene-, -alkylene-arylene-, -arylene-alkylene- or -alkylene-arylene-alkylene-.
   14. The process of any one of the preceding statements, wherein B is (2-5C)alkylene or arylene.
   15. The process of any one of the preceding statements, wherein B is ethylene, propylene or phenylene.
   16. The process of any one of the preceding statements, wherein C⁻ is a conjugate base of an acid, said acid having a pKa that is more negative than the acid capable of removing the acid-labile protecting group from the protected organic compound.
   17. The process of any one of the preceding statements, wherein C⁻ is a conjugate base of an acid, said acid having a pKa in acetonitrile lower than 12.65.
   18. The process of any one of the preceding statements, wherein C⁻ is the conjugate base of an acid selected from the group consisting of a phenolic acid, a carboxylic acid, a sulfonic acid or a phosphonic acid, said acids having a pKa that is more negative than the pKa of the acid capable of removing the acid-labile protecting group from the protected organic compound.
   19. The process of any one of the preceding statements, wherein C⁻ is selected from a phenolate, a carboxylate, a sulfonate, a thiosulfonate group and a phosphonate group.
   20. The process of any one of the preceding statements, wherein C⁻ is a sulfonate.
   21. The process of any one of the preceding statements, wherein C⁻ has the structure:

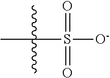 [SMILES: *S(=O)(=O)[O-]]

22. The process of any one of the preceding statements, wherein D⁺ is an organically-soluble cation.
23. The process of any one of the preceding statements, wherein D⁺ is an alkylammonium ion, an arylammonium ion, an N,N-alkylimidazolium ion, an N-alkylpyridinium ion, an alkylphosphonium ion, an arylphosphonium ion, an alkylarsonium ion or an arylarsonium ion.
24. The process of any one of the preceding statements, wherein D⁺ is an (2-4C)alkylammonium ion
25. The process of any one of the preceding statements, wherein D⁺ is a tertiary alkylammonium ion or a quaternary alkylammonium ion.
26. The process of any one of the preceding statements, wherein D⁺ is Et₃NH⁺, Et₄N⁺ or Bu₄N⁺.
27. The process of any one of the preceding statements, wherein the cation scavenger has a structure according to formula Ia:

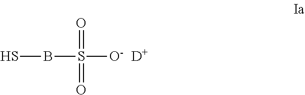

wherein B and D⁺ are as defined in any one of the preceding statements.
28. The process of any one of statements 1-26, wherein the cation scavenger has a structure according to formula Ib:

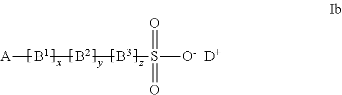

wherein
A and D⁺ are as defined in any one of the preceding statements;
B¹ is —CH₂—;
B² is arylene (e.g., phenylene);
B³ is —CH₂—;
x is 0-4;
y is 0 or 1; and
z is 0-4;
with proviso that at least one of x, y and z is not 0.
   29. The process of statement 28, wherein the sum of x and z is ≤5.
   30. The process of statement 28 or 29, wherein when y is 0, x and z are not 0.
   31. The process of statement 28 or 29, wherein when y is 1, x and z are 0-2.
   32. The process of statement 28, wherein (i) x is 2-4, and y and z are 0, or (ii) y is 1 and x and z are 0-1.
   33. The process of statement 28, wherein (i) x is 2-3, and y and z are 0, or (ii) y is 1, and x and z are 0.
   34. The process of any one of statements 1-26, wherein the cation scavenger has a structure according to formula Ic:

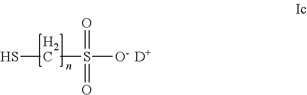

wherein D⁺ is as defined in any one of the preceding statements; and n is 2-8.
35. The process of statement 34, wherein n is 2-6.
36. The process of statement 34, wherein n is 2-3.
37. The process of any one of statements 1-26, wherein the cation scavenger has a structure according to formula Id:

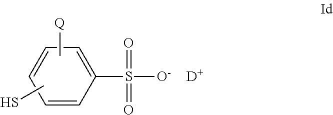

wherein D⁺ has any of the definitions outlined hereinbefore and Q is hydrogen or a substituent having a mass of <100 g mol⁻¹.
38. The process of statement 37, wherein the —SH group is meta or para to the —SO₃ group.
39. The process of statement 37 or 38, wherein Q is hydrogen.
40. The process of any one of statements 1-26, wherein the cation scavenger has a structure according to formula Ie, If or Ig:

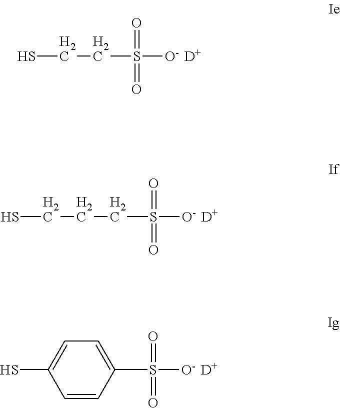

wherein D⁺ is as defined in any one of the preceding statements.
41. The process of any one of the preceding statements, wherein the acid capable of removing the acid-labile protecting group is a haloacetic acid, toluene sulfonic acid, hydrochloric acid, sulfuric acid, phosphoric acid or polyphosphoric acid.
42. The process of any one of the preceding statements, wherein the acid capable of removing the acid-labile protecting group from the protected organic compound is trifluoroacetic acid, dichloroacetic acid or trichloroacetic acid.
43. The process of any one of the preceding statements, wherein the acid capable of removing the acid-labile protecting group from the protected organic compound is trifluoroacetic acid.
44. The process of any one of the preceding statements, wherein the step of contacting the protected organic compound with the acid and the cation scavenger is conducted in at least one chlorinated hydrocarbon solvent or a polar aprotic solvent.
45. The process of any one of the preceding statements, wherein the step of contacting the protected organic compound with the acid and the cation scavenger is conducted in one or more of dichloromethane, acetonitrile, sulfolane, dimethylformamide, dimethylacetamide and dimethyl sulfoxide.
46. The process of any one of the preceding statements, wherein the step of contacting the protected organic compound with the acid and the cation scavenger is conducted in one or more of dichloromethane, acetonitrile and sulfolane.
47. The process of any one of statements 1 to 43, wherein the step of contacting the protected organic compound with the acid and the cation scavenger is conducted in at least one of ethyl acetate, tetrahydrofuran and toluene.
48. The process of any one of the preceding statements, wherein the protected organic compound is a nucleotide, amino acid or sugar.
49. The process of any one of statements 1 to 47, wherein the protected organic compound is a polymer or an oligomer.
50. The process of statement 49, wherein the protected organic compound is a defined monomer sequence polymer.
51. The process of any one of statements 1 to 47, 49 or 50, wherein the protected organic compound is an oligonucleotide, peptide, peptide nucleic acid or oligosaccharide.
52. The process of any one of the preceding statements, wherein the acid-labile protecting group is covalently bonded to a heteroatom (e.g., a N or O atom) of the organic compound.
53. The process of any one of the preceding statements, wherein the acid-labile protecting group is selected from the group consisting of dimethoxytrityl (Dmtr/DMT), tert-butyl (tBu), tert-butyl oxycarbonyl (Boc), mono-methoxytriphenyl (Mmtr), triphenyl methyl (Tr), pentamethyl dihydrobenzofuran sulfonyl (Pbf), Tetrahydropyranyl (Thp), tetrahydrofuranyl (Thf), para-methoxybenzyl (Pmb) and 2,4-dimethoxybenzyl.
54. The process of any one of the preceding statements, wherein the acid-labile protecting group is dimethoxytrityl (Dmtr/DMT).
55. The process of any one of statements 1 to 43, wherein the organic compound is a nucleotide or an oligonucleotide, the acid-labile protecting group is dimethoxytrityl (Dmtr/DMT) and the step of contacting the protected organic compound with the acid (e.g., TFA) and the cation scavenger is conducted in acetonitrile.
56. The process of any one of statements 1 to 43, wherein the organic compound is a defined monomer sequence polymer having a polyethylene glycol backbone, the acid-labile protecting group is dimethoxytrityl (Dmtr/DMT) and the step of contacting the protected organic compound with the acid (e.g., TFA) and the cation scavenger is conducted in dichloromethane.
57. The process of any one of statements 1 to 43, wherein the organic compound is an amino acid or a peptide, the acid-labile protecting group is triphenyl methyl (Tr) and the step of contacting the protected organic compound with the acid (e.g., TFA) and the cation scavenger is conducted in dichloromethane.
58. The process of any one of the preceding statements, wherein the acid and the cation scavenger are simultaneously contacted with the protected organic compound.
59. The process of any one of the preceding statements, wherein the acid and the cation scavenger are sequentially contacted with the protected organic compound.
60. The process of any one of the preceding statements, further comprising the step of isolating the organic compound, once deprotected.
61. The process of statement 60, wherein the organic compound, once deprotected, is isolated by membrane filtration (e.g., using a crosslinked polybenzimidazole membrane).
62. The process of statement 60, wherein the organic compound, once deprotected, is isolated by organic solvent nanofiltration.
63. A method for the preparation of a defined monomer sequence polymer by sequential coupling of monomeric units, wherein the method comprises one or more deprotection processes as defined in any one of the preceding statements.
64. The method of statement 63, wherein one end of the defined monomer sequence polymer is attached to a synthesis support during coupling of the monomeric units.
65. The method of statement 63 or 64, wherein the synthesis support is a branch point molecule.
66. The method of statement 63, 64 or 65, wherein the defined monomer sequence polymer is an oligonucleotide or a polymer having a polyethylene glycol backbone.
67. A cation scavenger as defined in any one of the preceding statements.

### EXAMPLES

One or more examples of the invention will now be described, for the purpose of illustration only, with reference to the accompanying figures:

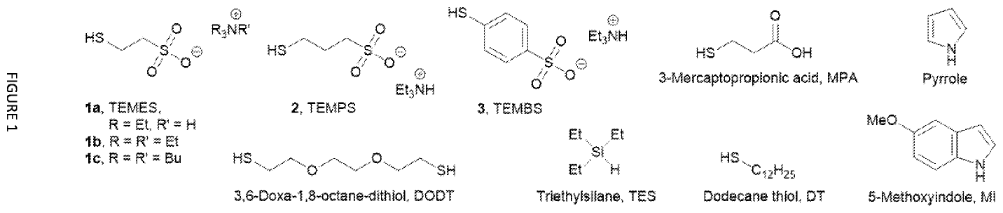

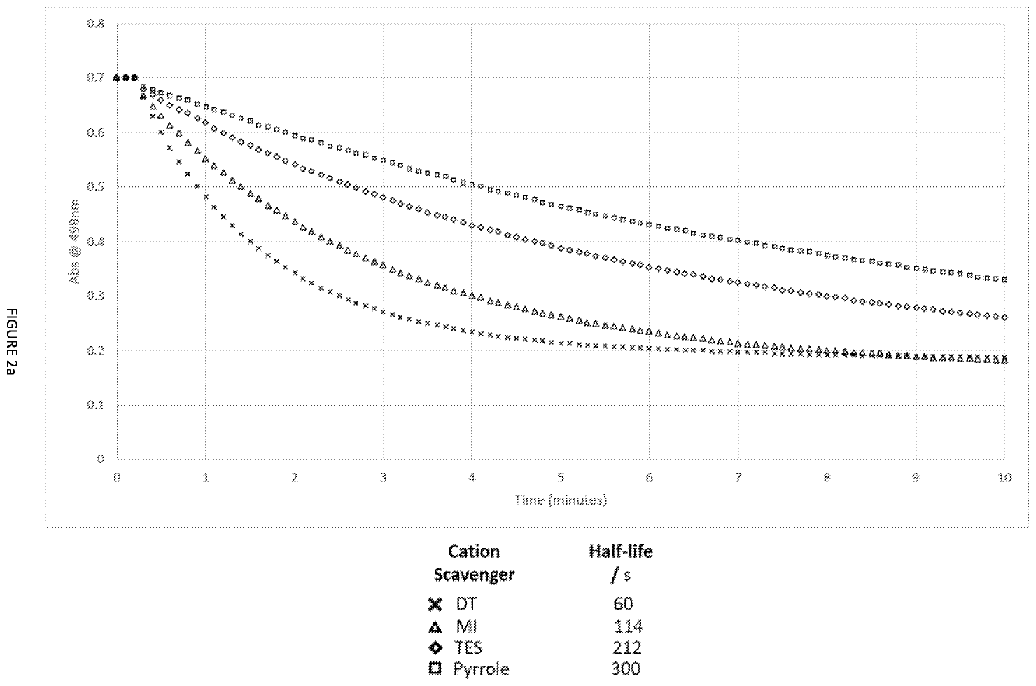

FIG. 1. Examples of cation traps forming part of the invention, 1-3, and commercially available cation traps.

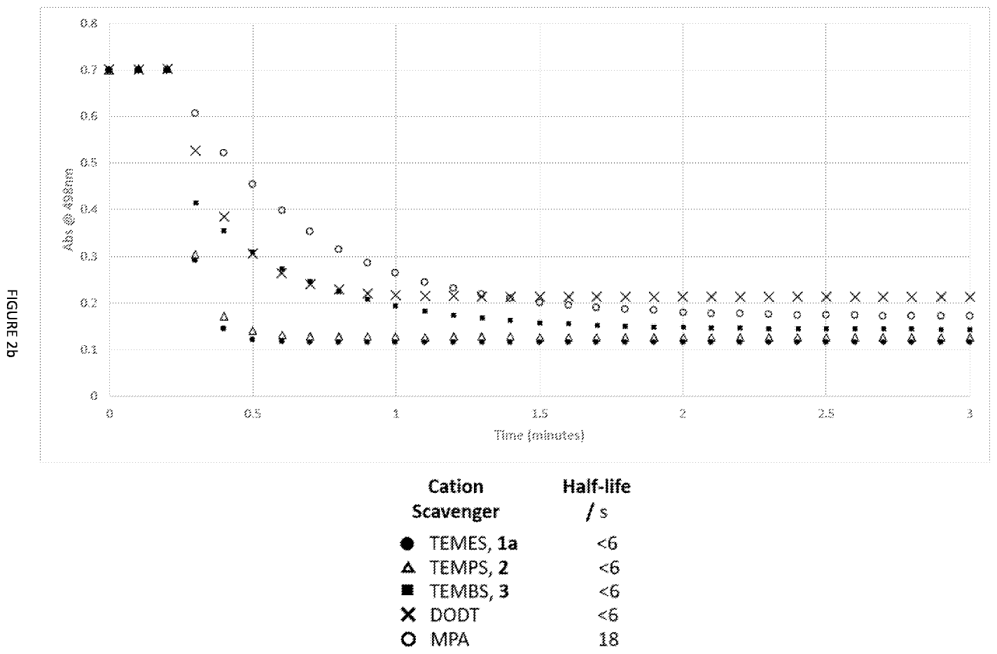

FIG. 2. Detritylation of Dmtr-hexamer-star 4 with 1 vol % TFA. After treatment of 4 with TFA for 20 min, allowing detritylation to equilibrate, a solution of cation scavenger was added to explore the speed and completeness of trapping: a) The performance of different cation scavengers, with poor trapping ability; b) the performance of different more effective cation scavengers, and this invention 1a, 2 and 3; c) the performance of scavenger 1 with different counter-ions.

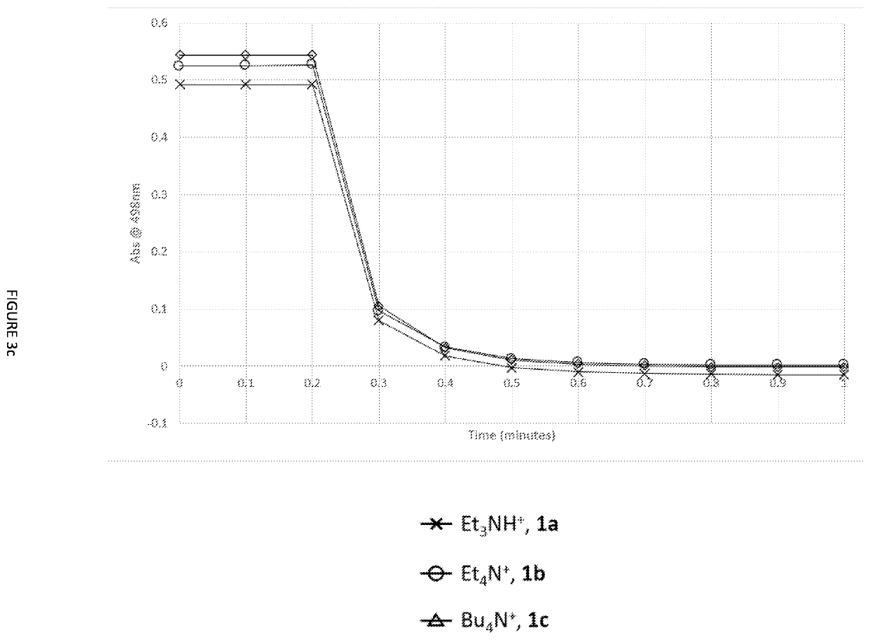

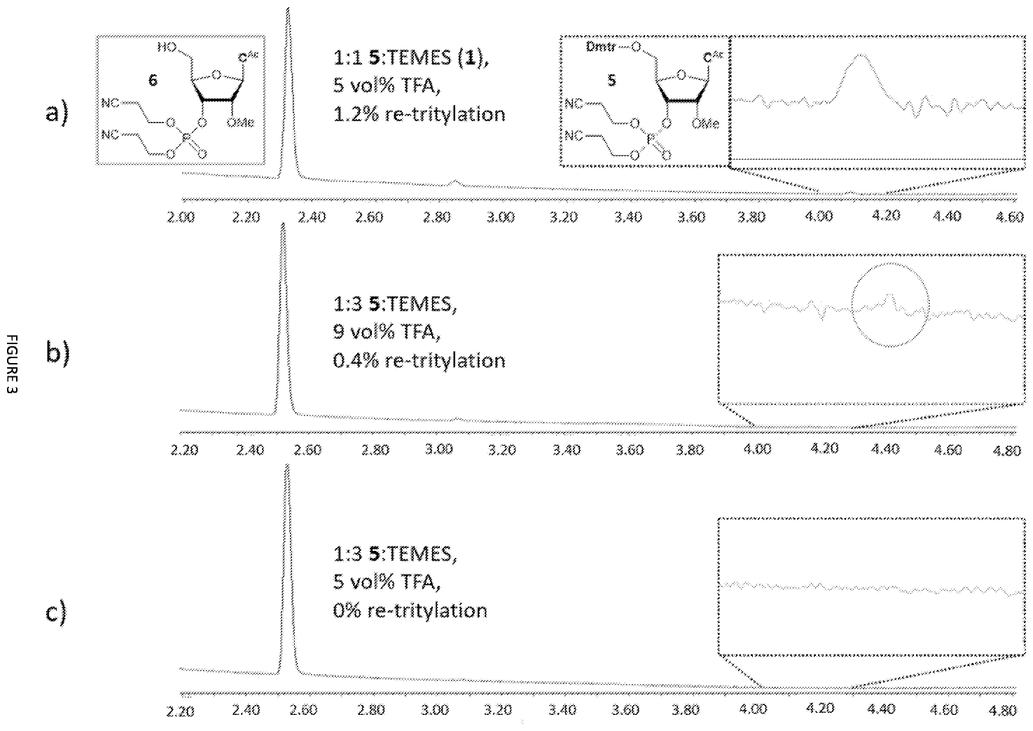

FIG. 3. Detritylation of 5′-Dmtr-3′-Me-N4-acetyl-cytidine 3′-dicyanoethyl phosphate (5). Liquid chromatograms (260 nm) after quenching with 3-picoline. Inset, expansion shows the degree of re-tritylation of product 6 with varying the ratios of substrate, acid and TEMES (1).

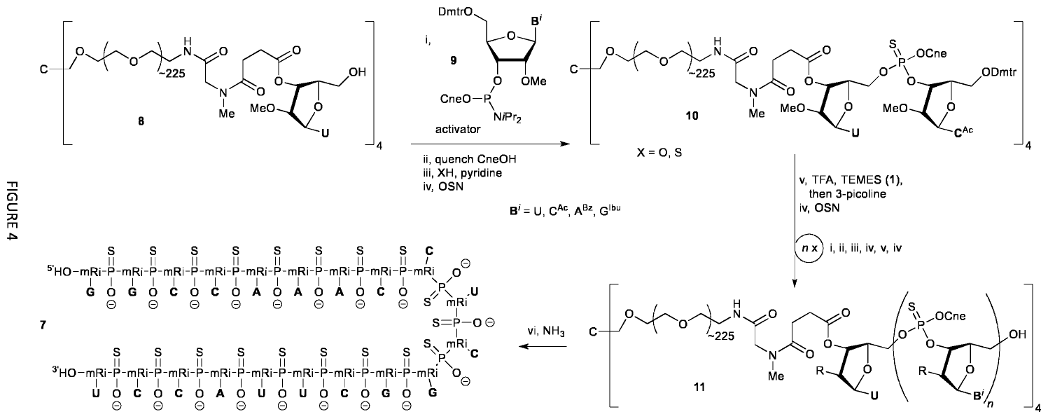

FIG. 4. Synthesis of M23D 20-mer ASO.

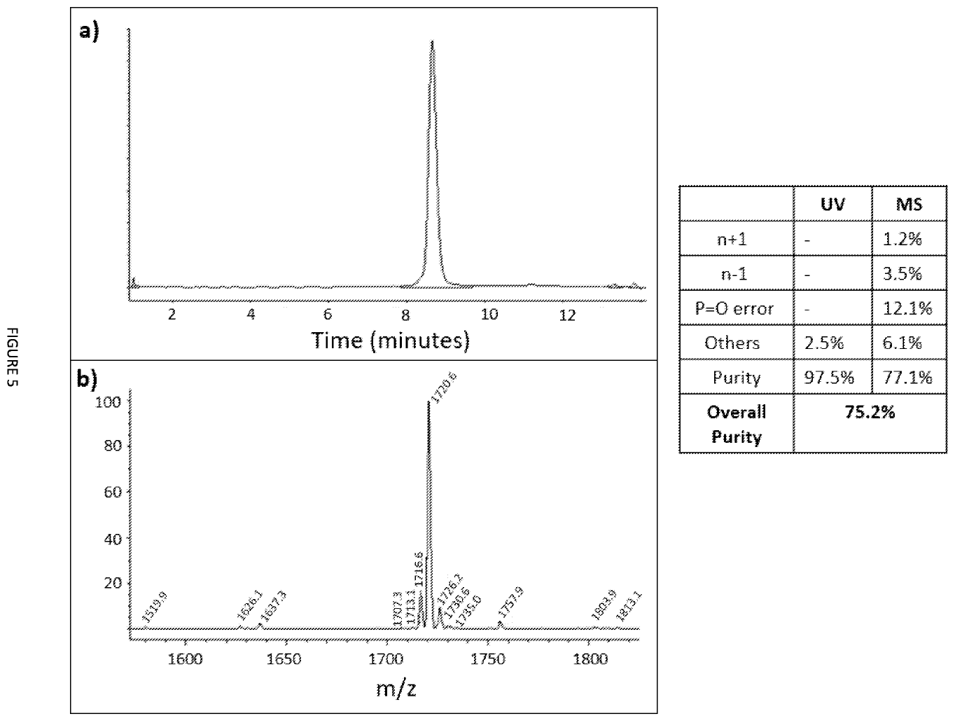

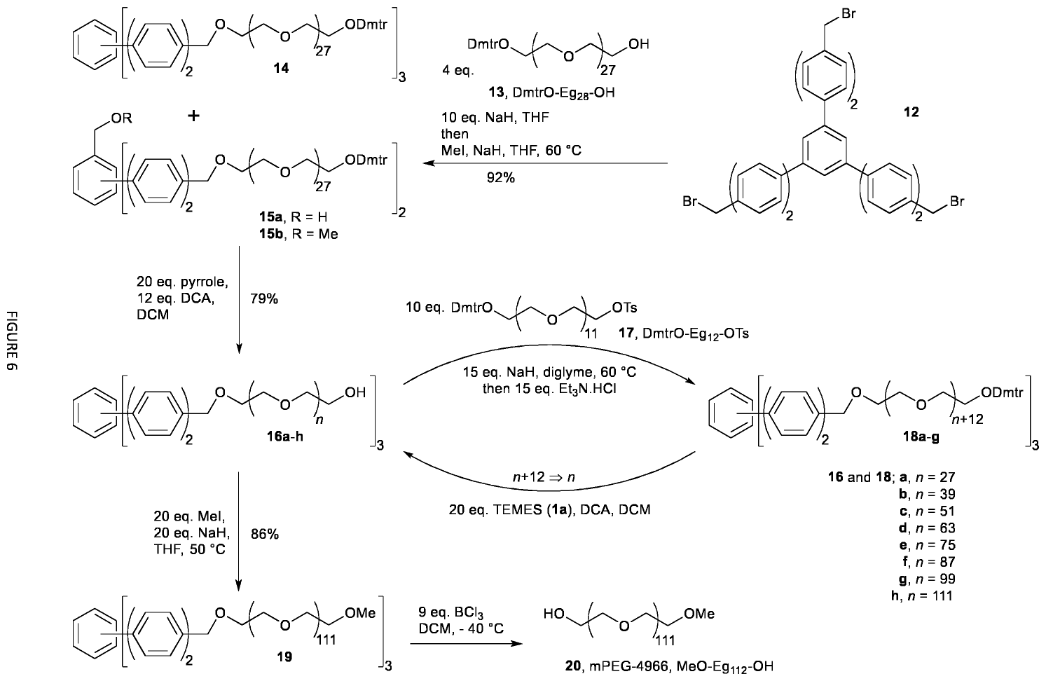

FIG. 5. LC-MS analysis of ASO 20-mer 7: a) diode array chromatogram; b) total ion current chromatogram.

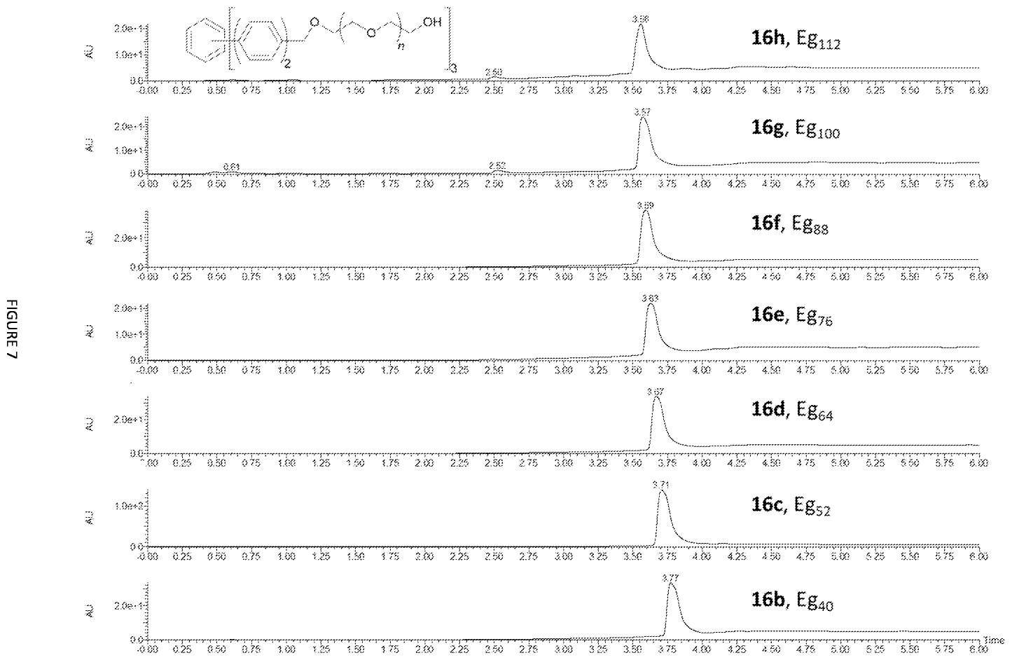

FIG. 6. Synthesis of mPEG-4966, 19.

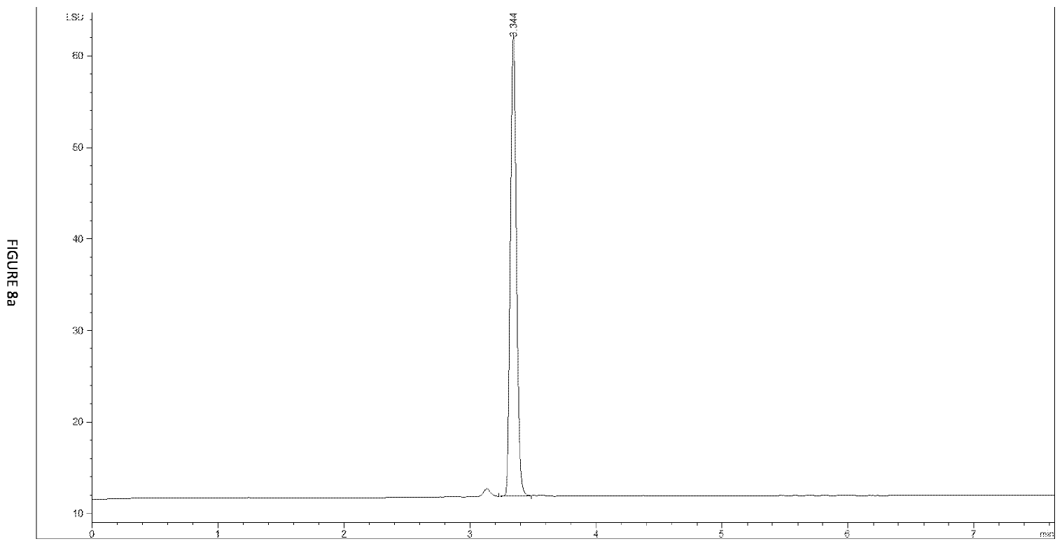

FIG. 7. UHPLC of mono-disperse HO-PEG-stars, 16: Eluted with a 40-95% 10 μM NH₄OAc—(MeCN-MeOH 4:1) gradient, with diode array detection. Notably, as the PEG chain becomes longer the retention time slowly decreases as the hydrophilic polymer chains mask the hydrophobic hub.

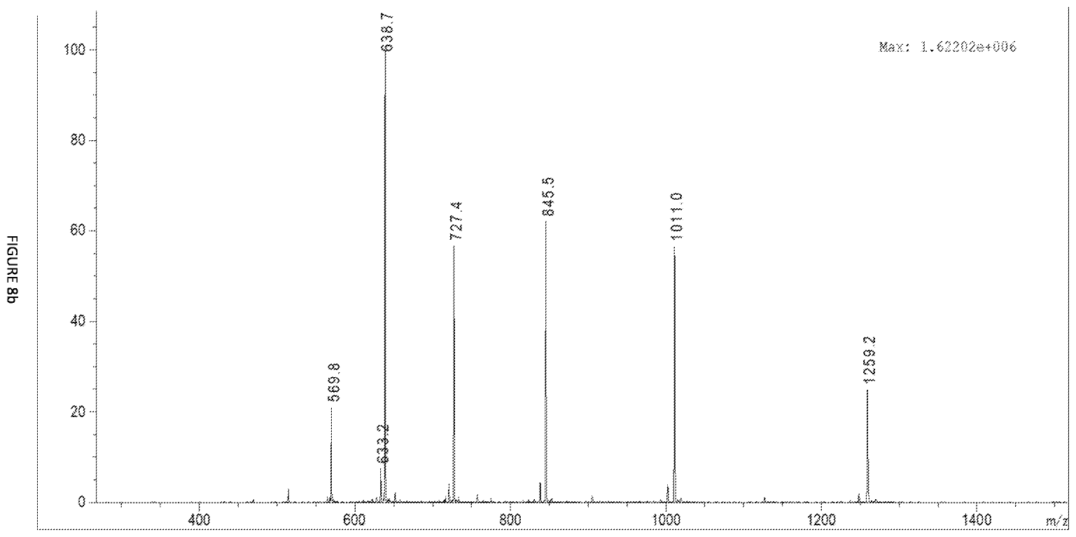

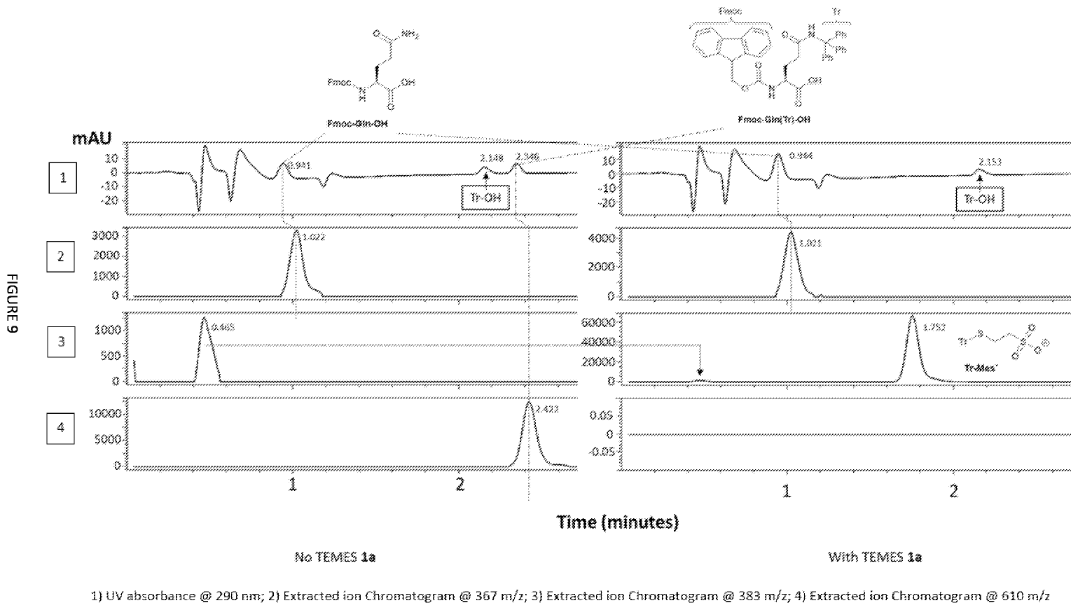

FIG. 8. a) UHPLC of MeO-Eg₁₁₂-OH, 20: Eluted with a 40-80% 10 μM NH₄OAc-MeCN gradient, with ELS detection (there were no UV-active solutes); retention time of 20=3.344 min; peak at 3.134 min (3% of main peak) is MeO-Eg₁₀₀-OH from incomplete coupling. b) ESI+MS of MeO-Eg₁₁₂-OH, 20: calculated m/z, [M+4·NH₄]⁴⁺=1259.3; [M+5·NH₄]⁵⁺=1011.0; [M+6·NH₄]⁶⁺=845.5; [M+7·NH₄]⁷⁺=727.3; [M+8·NH₄]³⁺=638.6; [M-Eg+8·NH₄]³⁺=633.1; [M+9·NH₄]⁹⁺=569.7. The full length Eg₁₁₂ sequence is contaminated with a small proportion of Eg₁₁₁ that is not resolved by LC, but can be detected in the MS, around 7% of the main peak height. This n−1 defect is the cumulative effect of Eg₁₁ in the Eg₁₂ monomer and base catalysed unzipping during chain extension.

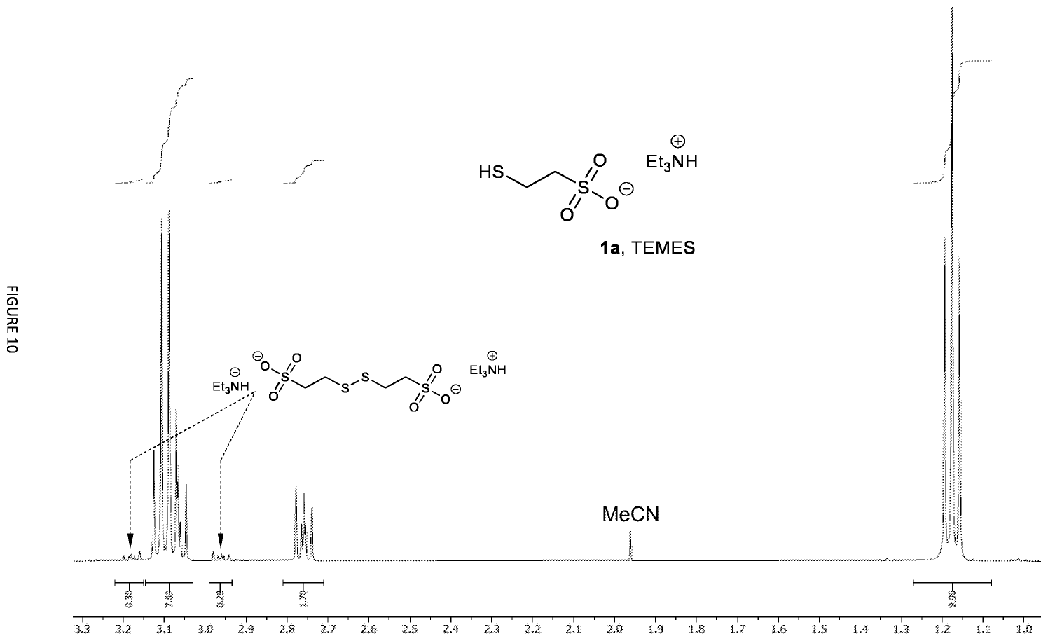

FIG. 9. LC-MS for detritylation of Fmoc-Gln(Tr)-OH in 5% TFA-DCM after 90 min: Left, with no trap; right plus TEMES 1a. For both chromatogram stacks, from top to bottom: Total diode array; negative mode extracted ion current (EIC) for Fmoc-Gln-OH, m/z=367±0.5; −ve mode EIC for Tr-Mes⁻, m/z=383−note, there is a low intensity (˜1000 counts) artefact with m/z=383 at 0.47 min (green arrow); −ve mode EIC for Fmoc-Gln(Tr)-OH, m/z=610.

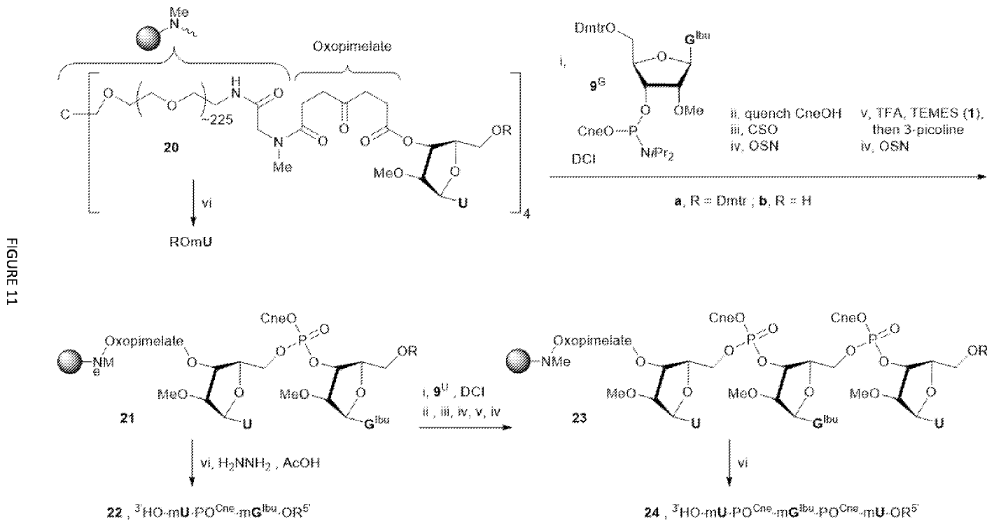

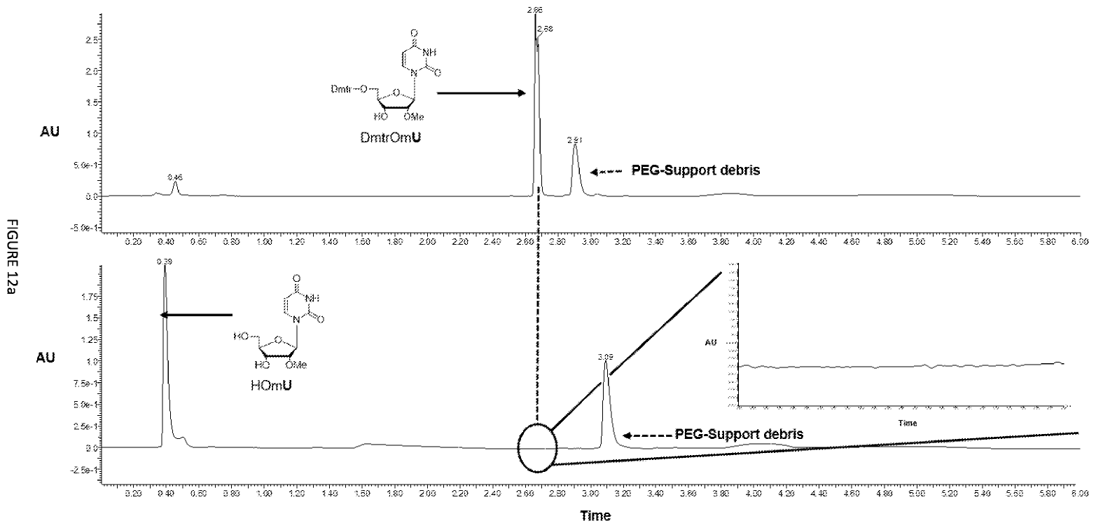

FIG. 10. ¹H NMR of TEMES 1a in D₂O.

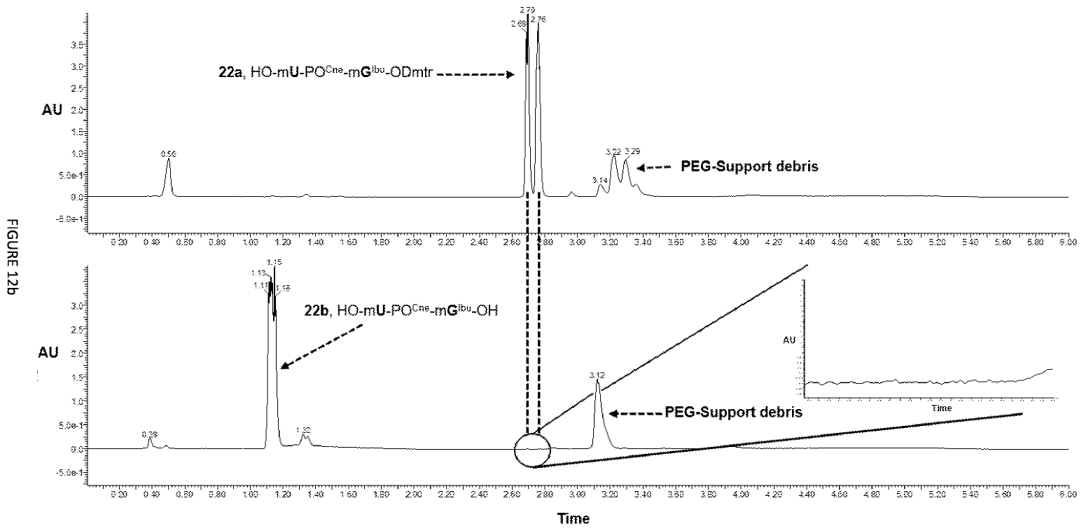

FIG. 11. Synthesis of mU-mG-mU trimer on a PEG-star support with a selectively cleavable oxopimelate linker, allowing hydrazine cleavage and analysis of fully protected oligos.

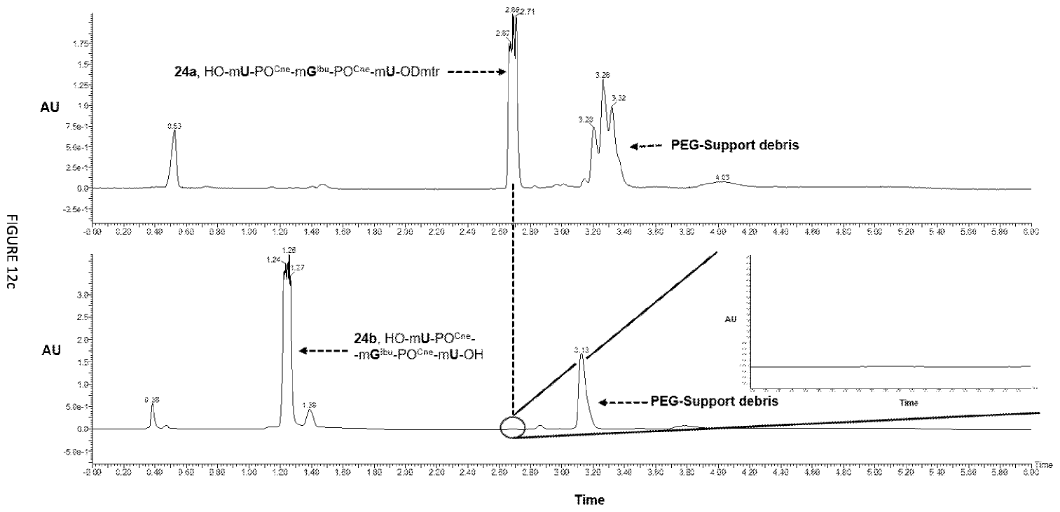

FIG. 12. UPLC traces of sequences cleaved from the oxopimelate-linked oligo-stars: a) mU nucleoside-star, top Dmtr on, bottom after detritylation; b) dimer-star, top Dmtr on, bottom after detritylation; c) trimer-star, top Dmtr on, bottom after detritylation.

### EXAMPLE 1

To Amberlyst-15 resin, proton form (300 g) in a 5 L conical flask cooled with a cold-water bath, were added first water (500 mL), then methanol (700 mL) and finally triethylamine (250 mL); these operations are exothermic. After mixing gently (to avoid breaking up the Amberlyst), an indicator strip was used to ensure the pH was around 10. The pH was again determined after 30 min as this neutralisation is not instantaneous; if the pH is neutral add more triethylamine.

The next day the neutralised Amberlyst was collected in a large funnel fitted with plug glass wool. The Amberlyst was washed first with water (2 L) and then with acetonitrile (2 L), discarding the filtrate. The washed beads were transferred to a 5 L conical flask to which acetonitrile (1.5 L), then sodium 2-mercaptoethansulfonate (MESNa, 50 g) dissolved in water (200 mL), were added. After gentle mixing, the conical flask was left to stand overnight.

The following day, the Amberlyst beads were filtered off in a large funnel fitted with a plug of glass wool, washing the beads extensively with acetonitrile (2 L). In a 2 L flask, the combined filtrate and washings were concentrated on a rotary evaporator, setting the water bath to 45 deg. C. Once liquid ceased to distil, the evaporation was repeated with further MeCN (3×100 mL). The oily residue was diluted with dry acetonitrile (100 mL) and 3 A molecular sieve (˜20 g) was added. After 1 h the suspension was filtered through a glass sinter funnel, then again through a 0.1 μm PTFA membrane. The filtrate was evaporated under reduced pressure and placed on high vacuum for 30 minutes to give triethylammonium mercaptoethanesulfonate (1a, TEMES, FIG. 1) as a viscous pale amber oil.

Cation scavengers 1b and 1c were prepared similarly to 1a, except the starting Amberlyst resin was neutralised with tetraethylammonium hydroxide and tetrabutylammonium hydroxide, respectively. Alternatively, 1c may be prepared by mixing tetrabutylammonium hydroxide with 2 eq. MESNa in water and extracting with DCM. After drying over sodium sulfate and evaporating the organic solvent, 1c is isolated in quantitative yield.

Triethylammonium 3-mercaptopropanesulfonate (TEMPS, 2), was prepared similarly to TEMES but using sodium 3-mercaptopropanesulfonate. Triethylammonium 4-mercaptobenzenesulfonate (TEMBS, 3), was prepared from a solution of 4-mercaptobenzene sulfonic acid in DCM which was neutralised with excess triethylamine and evaporated to dryness. All other cation traps are commercially available.

### EXAMPLE 2

A range of cation traps were tested under identical conditions to compare the performance of cation scavengers described in this invention to known cation scavengers. Acetonitrile was selected as the solvent because this solvent is used almost universally for oligonucleotide synthesis. A stock solution of Dmtr-hexanucleotide-star 4 in MeCN (0.1 mg/mL) was prepared.

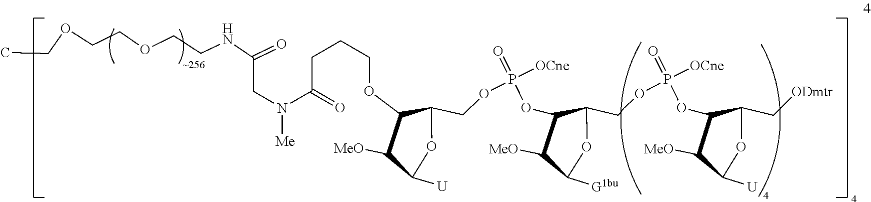

To a sample solution of 4 (1 mL) in a quartz cuvette was added TFA (10 μL), the solution was mixed, and the reaction left to stand for 20 min to equilibrate. Solutions of cation traps were prepared (100 mg/ml) in MeCN (1, 2, MPA, pyrrole, TES) or DCM (3, DT, MI). The cuvette was place in a UV-vis spectrophotometer, then an aliquot of cation trap was added, equivalent to 2.4×10⁵ mol, and the contents of the cuvette rapidly mixed. The disappearance of Dmtr⁺ cation was monitored at 498 nm, FIGS. 2a, 2b and 2c.

Dodecane thiol (DT) is a typical SH-based cation trap and consumed Dmtr⁺ cation smoothly with t_{1/2} of 1 min, but had only proceeded to 90% completion by the end of the experiment (10 min). DT scavenging of Dmtr⁺ cation was the fastest of the reported cation traps under these conditions. For 3-Mercaptopropionic acid (MPA, pKa˜4.3 in water, but >20 in MeCN), the acid moiety is less acidic that TFA (pKa in water 0.23), meaning that the carboxylic acid group will be protonated under the assay conditions. MPA performance was intermediate between 1-3 and DT.

3,6-Dioxa-1,8-octane-dithiol (DODT) is an SH-based cation trap (Harris and Brimble). It has a shorter half-life than DT for scavenging Dmtr⁺, similar to MPA, and proceeds to about the same level of completion as DT; notably, since it possesses two thiol moieties the comparison in FIG. 2b is at half the concentration of the other traps. However, it has the advantage of greater solubility in MeCN. In preparative studies, such as 5% TFA as used in examples below, it was observed that the trapping was more reversible than 1-3.

All three examples of this invention TEMES (1a), TEMPS (2) and TEMBS (3) were found to mop up the Dmtr⁺ cation extremely quickly and completely with half-lives<6 seconds. Notably, when the counter-ion of TEMES (1a) was exchanged for more hydrophobic species (1b and 1c), no change was observed in the rate of trapping of Dmtr⁺, FIG. 2b.

### EXAMPLE 3

It is notable with thiol/mercaptan (SH) based traps, such as 1-3 and DT, particularly in higher acid concentrations, that the initially intense orange colour of the Dmtr⁺ cation, although it becomes faint as scavenging proceeds, does not completely disappear. This indicates that the scavenging reaction, whilst fast and biased very heavily to the product side, is still an equilibrium

Therefore, experiments were conducted to identify under what conditions the invention worked best, FIG. 3. A solution of model trityl nucleotide, 5′-Dmtr-3′-Me-N4-acetyl-cytidine 3′-dicyanoethyl phosphate (5), in MeCN (10 mg/mL) was prepared. Also, solutions of TEMES (1) in MeCN (100 mg/mL), TFA in MeCN (5 vol %), and 3-picoline in MeCN (10 vol %) were made up. To the stock solution of 5 (1 mL) were added TEMES (1), then TFA solutions, and after a period of time 3-picoline was added to neutralise the TFA and quench any further reaction (Table 1). The solution was analysed by LC-MS.

TABLE 1
Volumes of reagent solutions added to 1 mL of stock solution 6.

| | TEMES, 1 | TFA | 3-Picoline | |
|---|---|---|---|---|
| Chromatogram | 10 wt | 5 vol | 10 vol | Time before |
| (FIG. 3) | % / μL | % / μL | % / μL | quench / min |
| a) | 36 | 50 | 100 | 30 min |
| b) | 108 | 90 | 180 | 20 min |
| c) | 108 | 50 | 100 | 30 min |

When, after detritylation of 5 to give 5′-OH nucleotide 6, there is no excess of TEMES (FIG. 3, chromatogram a), any residual Dmtr⁺ cation can be trapped by the product to reverse the reaction. Even with excess TEMES, when there is a higher concentration of acid, leading to a higher concentration of residual Dmtr⁺ cation when the picoline quench is added (FIG. 3, chromatogram b), then the exposed OH of 6 still manages to re-tritylate, although to a much lower degree. However, if the excess of TEMES is maintained, but the acid concentration is lowered (FIG. 3, chromatogram c), although the rate of detritylation is slower, when the reaction is quenched residual Dmtr⁺ cation is completely trapped by TEMES, with no re-formation of starting material 6. Thus, detritylation reactions conducted with a small excess of 2-4 equivalents of TEMES (1), approximately 50 mM in MeCN containing up to 5 vol % TFA, are likely to achieve complete reaction.

### EXAMPLE 4

FIG. 4 shows the synthesis of anti-sense oligonucleotide (ASO) 20-mer sequence 7 which was selected to test the application of the new cation scavengers in the preparation of a full sequence, performing detritylations under conditions similar to Example 3. Soluble synthesis support PEG-40k(SarH)₄ was condensed with Dmtr-mU succinate (2.5 eq. per arm) using dicyclohexyl carbodiimide (DCC) and hydroxybenzotriazole (HOBt), then transferred with filtration into a single-stage membrane separation synthesiser fitted with 5 circular cells (52 cm² each) of PBI18-DBX-MEA membranes (M-0, Oxley et al.), reaching a final oligo concentration of 10 mM. The crude material was diafiltered in neat acetonitrile to remove low MW debris, permeating 4 system volumes (or diavolumes, DV) of solvent with the system maintained at 30 deg. C. Detritylation was then performed within the synthesiser using 3 vol % TFA and TEMES (1, 15 eq. per PEG-star=3.75 eq. per arm). After 30 min the detritylation was quenched with 30 eq. pyridine, and diafiltration was resumed in neat acetonitrile (6 DV) until no remaining succinate building block could be detected to give pure PEG-40k(SarSuc-mU-OH)₄, 8.

Loaded nucleoside-star 8 was then subjected to one cycle of chain extension: Nucleoside phosphoramidite 9 (Bⁱ=C^{Ac}, 1.5 eq. per arm) was added to the synthesiser and activated with DCI (4 eq. per arm), then after 20 min the reaction was quenched with 2-cyanoethanol (CneOH, 3 eq. per arm). After 2 minutes sulfur transfer was initiated with xanthane hydride (XH, 3 eq. per arm) in pyridine. After 5 minutes, low MW reagent debris was removed by diafiltration (4 DV), and the temporary 5′-Dmtr protecting group was then unblocked as above with 3 vol % TFA (thermostated at 30 degrees C.) and TEMES (1, 3.75 eq. per arm=2.5 eq. per Dmtr, including excess phosphoramidite 9). The detritylation was quenched with excess pyridine, and diafiltration continued (6 DV, average permeate flux 33 mL/min) until no building block debris remained to give pure dinucleotide-star 10.

This cycle was repeated, with reagents injected into the synthesiser in the order required to build up ASO sequence 7, removing excess reagents and debris by OSN as above. As the length of the oligo on the PEG-star support increased, the permeate flux dropped slowly from 30 mL/min at trimer-star to 7.9 mL/min at 12-mer-star. The amount of remaining building block debris at the end of the synthesis cycle also rose to 2% at 8-mer-star and finally became unacceptable with 17% building block debris remaining at the end of diafiltration of 12-mer-star. Therefore, after 12-mer coupling to 13-mer-star the membranes were changed to PBI16-DBX-MEA (M-0, Oxley et al.) and synthesis of the sequence continued to full length ASO with good fluxes (dropping from 23 to 12 mL/min at 20-mer) and wash-out of building block debris being complete up to 17-mer, and only traces remaining in the final three cycles.

After detritylation of the 20-mer-star, the final oligo-star was washed from the synthesiser, rinsing with MeCN, and the solvent was evaporated under reduced pressure. A sample of the crude oligo-star was taken up in dimethylamine-DMF 3:7 v/v, and after 30 minutes the solvent was stripped off. The residue was taken up in conc. ammonia and heated in a sealed tube at 55 deg. C. overnight. The following day the solvent was stripped off and the residue evaporated from MeCN three times to remove residual water. Finally, the residue was triturated with MeCN and the suspension centrifuged at 6000 rpm for 30 min. The pellet was collected and analysed by ion-pair reversed phase LC-MS to reveal the full-length 20-mer ASO 7 in 75% UV-purity, FIG. 5.

### EXAMPLE 5

FIG. 6 shows the stepwise synthesis of mono-disperse mono-methoxy poly(ethylene glycol) (mPEG) 20. A key aspect of this process is the detritylation of the growing PEG terminus during each chain extension cycle using the cation trap TEMES, 1a, in dichloromethane (DCM).

### Synthesis of Starting Eg₂₈-Star, 16a

DmtrO-Eg₂₈-OH (13, 6.21 g, 4 eq.) was evaporated from MeCN (3×70 mL), then taken up in THF (14 mL), and NaH (60% dispersion in mineral oil, 165 mg, 4 eq.) was added. After stirring for 5 min, trivalent hub-tribromide 12 (866 mg, 1.0 mmol) was added and the reaction stirred for 3 hr; following by LC-MS, if the reaction fails to initiate, it may be warmed up to 50° C. until 12 dissolves, when the reaction will proceed rapidly. The mixture was then cooled to room temp., and conc. ammonium chloride was carefully added dropwise under argon until the fizzing ceased, then the solvent was evaporated. The residue was purified by flash chromatography through a column of silanised silica (9 cm diameter, 10 cm deep): the crude material was dry loaded onto silanised silica (8 mm depth), and the column eluted with a gradient of water-acetone. The product eluted in 65-80% acetone, and the organic solvent was evaporated from the appropriate fractions at reduced pressure. The aqueous residue was saturated with NaCl and extracted with chloroform (×4). The combined organic layers were dried over Na₂SO₄, and evaporated to dryness to give tris-DmtrO-Eg₂₈-star 14 (4.794 g, 92%).

Protected PEG-star 14 contains approximately 1% of 2-arm contaminant 15a from adventitious water in the preceding loading reaction. Therefore, this site was methylated prior to detritylation to simplify purification of 16a and to ensure that any byproduct carried through will not affect final mPEG quality. PEG-star 14 (4.794 g, 0.917 mmol) was dissolved in THF (14 mL) to which was added NaH (60% dispersion in mineral oil, 163 mg, 4.4 eq.) then Mel (0.25 mL, 4.4 eq.), and the reaction was stirred at 60° C. overnight; LC-MS showed that all 15a had been converted to 15b. The following day, after cooling to room temp., conc. NH₄Cl was carefully added dropwise under argon until fizzing ceased. The solvent was then evaporated, the residue dispersed in chloroform and partitioned with saturated NaHCO₃. The aqueous layer was back-extracted with chloroform (×3), the combined organic layers were dried over Na₂SO₄, and the solvent stripped off under reduced pressure.

The crude residue (5.052 g) was dissolved in DCM (30 mL) to which was added pyrrole (1.39 mL, 20 eq.) then dichloroacetic acid (DCA, 0.99 mL, 13 eq.). After 45 min all traces of orange colour had dissipated, and the acid was neutralised with half-saturated NaHCO₃. After dilution with chloroform, the layers were partitioned, and the aqueous layer back-extracted with more chloroform (×4). The combined organic layers were dried over Na₂SO₄, and the solvent stripped off under reduced pressure. The residue was purified by flash chromatography through a column of normal phase flash silica (6 cm diameter, 5 cm deep): the column was eluted with a gradient of methanol-chloroform; the product eluted 4-8% methanol. The appropriate fractions were combined and evaporated at reduced pressure to provide HO-Eg₂₈-star 16a (n=27, 3.449 g, 79%).

Chain Extension Cycle from Eg₂₈ to Eg₁₁₂

The hydroxy-PEG-star 16 was subjected to repeated rounds of chain extension with Eg₁₂ building block 17, followed by detritylation, up to a total length of Eg₁₁₂. Quantities and yield for each step are given in Table 2. The general procedure is outlined below.

TABLE 2
Reagent quantities and yields for cycles of mono-disperse PEG-star chain extension.

| Starting | | | | | | | | | | Product star, | | |
|---|---|---|---|---|---|---|---|---|---|---|---|---|
| star, 16a-g | | Eg₁₂ | 60% | | | | TEMES | | | 18a-g | | |
| n | g | 17/g | NaH/mg | Diglyme/mL | Et₃N•HCl/g | Time/hr | (1a)/g | DCA/mL | DCM/mL | n | g | % |
| a, 27 | 2.28 | 5.05 | 302 | 15 | 1.04 | 40 | 2.45 | 0.84 | 40 | 39 | 2.95 | 99 |
| b, 39 | 2.90 | 4.91 | 293 | 15.5 | 1.01 | 40 | 2.32 | 0.80 | 40 | 51 | 4.48 | 95 |
| c, 51 | 3.43 | 4.60 | 284 | 18 | 0.95 | 42 | 2.23 | 1.14 | 40 | 63 | 4.01 | 96 |
| d, 63 | 3.98 | 4.40 | 258 | 17 | 0.91 | 40 | 2.132 | 1.09 | 35 | 75 | 4.52 | 97 |
| e, 75 | 4.47 | 4.20 | 252 | 17 | 0.87 | 72 | 2.04 | 1.40 | 35 | 87 | 4.71 | 92 |
| f, 87 | 4.65 | 3.81 | 228 | 17 | 0.79 | 67 | 1.85 | 1.26 | 32 | 99 | 5.01 | 95 |
| g, 99 | 4.96 | 3.60 | 220 | 17 | 0.74 | 66 | 1.68 | 1.18 | 30 | 111 | 5.31 | 96 |

Combined HO-Eg₂₈-star 16 and DmtrO-Eg₁₂-OTs 17 were co-evaporated from anhydrous MeCN (3×75-100 mL), then transferred to a PFA flask in several aliquots of anhydrous THF, and the solvent was again evaporated. The residue was dissolved in anhydrous diglyme, then a 60% dispersion of NaH in mineral oil was added and the reaction stirred briskly at 60° C. for at least 40 hr; LC-MS frequently showed an induction period of up to 3 hours, but the reaction was usually complete after stirring overnight.

After cooling to room temp., a solution of triethylammonium chloride in MeCN-water 3:1 (6-7 mL) was added. Once fizzing had ceased the mixture was evaporated from MeCN (×2), then TEMES 1a dissolved in MeCN was added to the mixture and the solvent was stripped off again. Finally, the residue was dispersed in DCM and DCA was added to the mixture. The detritylation reaction was monitored by LC-MS and when complete (40-60 min) was neutralised with triethylamine. The solvent was evaporated, and the residue diluted into methanol.

The crude product 18 was purified by diafiltration in methanol containing 0.2% conc. NH₃, over PBI18-DBX-J1000 membranes (M-100, Oxley et al.). The crude solution was transferred to the diafiltration apparatus with paper filtration in MeOH. The 2-stage membrane diafiltration apparatus consisted of 4 flat sheet cells in the first stage, and 2 in the second stage, each cell 52 cm², with gear pumps providing the cross-flow in each stage. The first stage had a volume of ca. 230 mL, and the second stage 120 mL, with permeate from the second stage going to waste. A recycle ratio of approximately 0.5 was used, returning solvent from the second stage to the first. The system was pressurised to 16 bar in the first stage, and 7 in the second, with the pressure in each circulation loop controlled by pressure relief valves. For every chain extension a total of 3 L of solvent was permeated, with an average permeate flux from the second stage of ca. 11 mL/min. When diafiltration was complete, the retentate was efficiently washed out from both stages with further MeOH, and evaporated to dryness to give hydroxy-PEG-star 18 extended by 12 ethylene glycol units, Eg₁₂, FIG. 6. This material was used directly in the next round of chain extension.

Terminal Methylation and Cleavage from Hub

HO-Eg₁₁₂-star 16h (4.246 g, 0.275 mmol) was evaporated from anhydrous MeCN (3×45 mL) and transferred to a PFA flask in anhydrous THF (20 mL). The solvent was evaporated and the material evaporated again from THF (2×5 mL). The residue was taken up in THF (9 mL) to which was added NaH (60% dispersion in mineral oil, 220 mg, 20 eq.), immediately followed by Mel (0.34 mL, 20 eq.). With vigorous stirring, the reaction was then warmed to 50° C. and stirred for a further 14 hr. The next day, the suspension was cooled to room temp., and a solution of triethylammonium chloride (756 mg, 20 eq.) in MeCN-water 4:1 (2.5 mL) was carefully added dropwise under argon until fizzing ceased, after which the solvent was evaporated. The crude material was purified as above by diafiltration in MeOH to provide the methylated Eg₁₁₂-star 19 (3.656 g, 86%) as a light brown waxy solid.

MeO-Eg₁₁₂-star 19 (3.603 g, 0.233 mmol) was dissolved in DCM (50 mL) and the well stirred solution was cooled to −70° C. in in a Cardice-acetone bath. A first aliquot of boron trichloride in DCM solution (1M, 3×2.33 mL, 30 eq.) was added dropwise to the solution, then the solution was allowed to warm to −40° C.; the solution darkens with precipitate forming as the BCl₃ is added, but returns to a pale colour upon warming. The reaction was incomplete by LC-MS after the first aliquot of BCl₃ had been warmed to −40° C., so the reaction was re-cooled to −70° C. and the process repeated with another portion of BCl₃; three doses of BCl₃ were required for the reaction to reach completion. The mixture was re-cooled to −70° C., then MeOH (20 mL) was added dropwise, followed after 10 min by solid NaHCO₃ (3.5 g, 42 eq.), and finally the suspension was allowed to warm to room temp.

The solvent was evaporated, and the residue suspended in MeOH. The mixture was then filtered and the solvent once more evaporated to provide a crude gum (5.903 g). This material was purified as above by diafiltration in MeOH; notably, the UV-active hub debris permeated. After washing out the apparatus, and evaporating the combined permeate from both stages, MeO-Eg₁₁₂-OH (20, 3.290 g, 95%) was isolated as a pale cream coloured waxy solid (see FIG. 7).

### EXAMPLE 6

A stock solution of Fmoc-Gln(Tr)-OH (1 mg/mL) was made up in DCM. To one 1 mL aliquot was added TFA (50 μL), and to another was added both TFA (50 μL) and TEMES (1a, 30 L, 7.5 eq.). After 90 min the reaction was quenched with pyridine (50 μL) and analysed by LC-MS, FIG. 9; only with TEMES (FIG. 9, right; bottom chromatogram exhibits no trace of starting material) does the reaction proceed to completion.

### EXAMPLE 7

Improved TEMES synthesis: AmberChrom resin (hydrogen form, 600 g, 1.28 mol) was placed in a 5 L conical flask. Cold water (1000 mL) was added, followed by methanol (1400 mL); this and the subsequent operation are exothermic. Triethylamine (5×100 mL aliquots) was added to the flask, swirling the flask after each addition. This procedure avoids the need to cool the reaction flask. The AmberChrom suspension was then thoroughly mixed, and after 30 min the pH was checked with an indicator strip to ensure it was ˜10; neutralisation is not instantaneous, but if after 30 min the pH is still neutral add more triethylamine. The mixture was left to stand in a fume hood overnight.

The next day the neutralised AmberChrom was collected in a large sinter funnel and washed with water (2×1 L), discarding the filtrate. Sodium 2-mercaptoethansulfonate (MESNa) (100 g, 0.61 mol) was dissolved in water (250 mL) and transferred to a 5 L conical flask. To this was added a portion resin (˜100 g) to form a slurry, and the flask was swirled for ˜2 mins to promote initial cation exchange between MESNa and the resin. Acetonitrile (50 mL) was added to the flask, followed by further resin (˜100 g), again swirling for ˜2 mins. This step was repeated once more. Next, acetonitrile (100 mL) was added to the slurry, followed by more resin (˜200 g), once more swirling the mixture for ˜2 mins; this step was repeated until the resin was consumed. Finally, further acetonitrile (1200 mL) was added to the suspension, the flask was swirled for ˜1 min, and then left to stand overnight under the fume hood.

The following day, the supernatant liquid was collected, filtering off the spent AmberChrom beads in a sinter funnel, and washing the resin with acetonitrile (2×1 L). The combined filtrate and washings were evaporated in a rotary evaporator (in at least a 2 L flask), using a warm heating bath (45 deg. C.). Once liquid distillation ceased, the collection flask was emptied and evacuation continued; emptying the collection flask reduces the vapour pressure inside the evaporator allowing more liquid to distil. At the end of the evaporation ensure the rotating flask is spinning slowly to allow the thick oil to turn over; this maximises evaporation of the azeotrope, exposing new surface area to vacuum.

Preparing anhydrous TEMES: The residual oily TEMES was re-evaporated from anhydrous MeCN (<30 ppm of water content; 3×150 mL); after the first azeotrope, charging MeCN for the second or third azeotrope may result in a cloudy mixture. Next, further anhydrous acetonitrile (1.2 L) and a molecular sieve bag (Oligo Solution, 10 Gram Molecular Sieve Solvent Pouch™) was added. The cloudy mixture was stirred over the weekend, and then quickly filtered through a sinter funnel; a small amount of starting MESNa remains which is insoluble in completely anhydrous MeCN. The filtrate was then re-filtered through a 0.22 μm PTFA membrane using a steel pressure filter holder; the pressure was set to 1 bar, but a maximum 3 bar can be used. The filtrate was evaporated under reduced pressure and placed under high vacuum for ˜6 hours to obtain TEMES as a pale-yellow viscous oil (129 g).

¹H NMR (D₂O, see FIG. 10): δ=3.20-3.16 (0.3H, m, disulfide CH₂—SO₃—), 3.12-3.15 (7.7H, m, 3×NCH₂Me+Mes CH₂—SO₃—), 2.98-2.94 (0.3H, m, disulfide CH₂—SH), 2.78-2.74 (1.7H, m, Mes CH₂—SH), 1.18 (9H, t, J=9 Hz, 3×NCH₂Me) ppm.

### EXAMPLE 8

To definitively demonstrate that quantitative detritylation occurs using TEMES 1a as the Dmtr⁺ trapping reagent, a 4-oxoheptanedioate linker was installed between the PEG-support and the growing oligonucleotide, FIG. 11. This strategy allows cleavage of the intact, protected oligo from the support, and therefore for detailed LC-MS analysis of the growing oligo products. The synthesis was conducted similarly to Example 4.

Soluble synthesis support PEG-40k(SarH)₄ was condensed with Dmtr-mU 3′-oxopimelate (2.5 eq. per arm) using DCC (10 eq.) and HOBt (10 eq.), then transferred with filtration into a single-stage membrane separation synthesiser fitted with 5 circular cells (52 cm² each) of PBI17-DBX-Jeffamine membranes, reaching a final concentration of 8 mM nucleoside. The crude material was diafiltered in neat acetonitrile, permeating 4 DV of solvent with the system maintained at 30 deg. C. Detritylation of Dmtr-mU-star 20a was then performed within the synthesiser using 5 vol % TFA and TEMES (1a, 27 eq.=2.7 per Dmtr from the oxopimelate). After 30 min the detritylation was quenched with 3-picoline (10 vol %), and diafiltration was resumed in neat acetonitrile (6 DV) until no remaining oxopimelate building block could be detected to give pure PEG-40k(SarOpim-mU-OH)₄, 20b.

At the end of DF1 and before starting DF2, a 1 mL sample was withdrawn from the membrane reactor and taken for cleavage analysis. Hydrazinium acetate (10 eq.), freshly prepared from hydrazine monohydrate (120 μL) and acetic acid (200 μL), was added to the DF1 and DF2 samples. After incubating for 2 hr at 25 deg. C., an aliquot from each sample was diluted 1000-fold and analysed by LC-MS, FIG. 12a. The sample from DF1, FIG. 12a top trace, shows almost no detectable detritylated 2′-methyl uridine (HOmU), whereas there is no detectable DmtrOmU in the lower diode array trace from DF2. The limit of detection by MS was lower than UV-vis, and was determined to be <0.01%. No HOmU could be detected in the DF2 sample by MS.

Loaded nucleoside-star 20b was then subjected to one cycle of chain extension: Nucleoside phosphoramidite 9^{G} (1.5 eq. per arm) was added to the synthesiser and activated with DCI (4 eq. per arm), then after 20 min the reaction was quenched with CneOH (3 eq. per arm). After 2 minutes, oxidation was initiated with (1S)-(+)-(10-camphorsulfonyl)-oxaziridine (CSO, 36 eq.) in MeCN. After 1 hr, low MW reagent debris was removed by diafiltration (4 DV), and Dmtr-dimer-star 21a was then unblocked as above with 5 vol % TFA and TEMES (1a, 18 eq.=3 eq. per Dmtr from the phosphoramidite). The detritylation was quenched with 3-picoline (10 vol %), and diafiltration continued (6 DV) until no building block debris remained to give pure dinucleotide-star 21b.

As with loaded nucleoside-star 20, samples of dimer-star 21 were taken both at the end of DF1 and before starting DF2, and subjected to selective hydrazinolysis. LC-MS analysis, FIG. 12b demonstrated that the detritylation was complete, with no Dmtr-dimer 22a detectable in 3′-HO-dimer 22b either by UV-vis or MS.

Another cycle of chain extension was undertaken, as above, but now with uridine phosphoramidite 9^{U}. Again, the trimer-star 23 was subjected to selective hydrazinolysis before and after detritylation in the membrane synthesiser, FIG. 12c. It was once more found impossible to detect any trace of incompletely detritylated trimer 24a in the sample of 3′-hydroxy trimer 24b extracted from the synthesiser immediately after completing TEMES mediated 5′-unblocking. It is therefore conjectured that any n−1 deletion sequences derived from oligos detritylated in the presence of the TEMES (1a) cation scavenger do not derive from incomplete detritylation.

While specific embodiments of the invention have been described herein for the purpose of reference and illustration, various modifications will be apparent to a person skilled in the art without departing from the scope of the invention as defined by the appended claims.

### BIBLIOGRAPHY

Reese and Pei-Zhou, J. Chem. Soc., Perkin Trans. 1, 1993, 2291-2301
US 2015/0080565
US 2018/0282365
Shi et al., J. Org. Chem., 2022, vol 87, pages 2087-2110
U.S. Pat. No. 5,714,597A
Lutz J. F., Ouchi M., Liu D. R., Science 2013, 341, 1238149-1
Hartmann L., Borner H. G., Adv. Mater. 2009, 21, p 3423
Dong R., Liu R., Gaffney P. R. J., Schaepertoens M., Marchetti P., Williams C. M., Chen R. and Livingston A. G. Nature Chemistry (2019) 11 pp. 136-145
PCT/GB2007/050218
PCT/GB2015/050179
U.S. Pat. No. 10,913,033
U.S. Pat. No. 6,683,173
Peacock H. et al., J. Am. Chem. Soc. (2011), 133, 9200
Oxley, Gaffney, Kim, Marchetti, Livingston, J. Membrane Sci., 2022, vol 647, p 120199
Harris P. W. R., Brimble M. A. et al. J. Pep. Sci. 2014, vol 20, 186-190
Samson D., Minuth M., Improving Oligonucleotide Manufacture with Stirred-bed Technology, White Paper, October 2023.

1. A process for deprotecting an organic compound, the process comprising the step of contacting a protected organic compound comprising an acid-labile protecting group with:
(i) an acid capable of removing the acid-labile protecting group from the protected organic compound, and
(ii) a cation scavenger having a structure according to formula I: 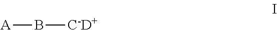
wherein
A is a group capable of forming a covalent bond with the acid-labile protecting group once removed from the organic compound;
B is absent or is a linking moiety;
C⁻ is a negatively charged group; and
D⁺ is a counter ion.

2. The process of claim 1, wherein A is a nucleophilic group.

3. The process of claim 1 or 2, wherein A is a sulfhydryl, phenolyl, anisolyl, thioanisolyl, electron-rich aryl or silyl.

4. The process of claim 1, 2 or 3, wherein A is sulfhydryl.

5. The process of any one of the preceding claims, wherein B is a linking moiety separating A from C⁻ by a distance of 2-20 bond lengths.

6. The process of any one of the preceding claims, wherein B is a linking moiety comprising at least one of an alkylene group and an arylene group (e.g., phenylene).

7. The process of any one of the preceding claims, wherein B is ethylene, propylene or phenylene.

8. The process of any one of the preceding claims, wherein C⁻ is a conjugate base of an acid, said acid having a pKa that is more negative than the acid capable of removing the acid-labile protecting group from the protected organic compound.

9. The process of any one of the preceding claims, wherein C⁻ is the conjugate base of an acid selected from the group consisting of a phenolic acid, a carboxylic acid, a sulfonic acid or a phosphonic acid, said acids having a pKa that is more negative than the pKa of the acid capable of removing the acid-labile protecting group from the protected organic compound.

10. The process of any one of the preceding claims, wherein C⁻ has the structure:

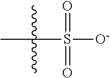 [SMILES: *S(=O)(=O)[O-]]

11. The process of any one of the preceding claims, wherein D⁺ is an alkylammonium ion, an arylammonium ion, an N,N-alkylimidazolium ion, an N-alkylpyridinium ion, an alkylphosphonium ion, an arylphosphonium ion, an alkylarsonium ion or an arylarsonium ion.

12. The process of any one of the preceding claims, wherein D⁺ is Et₃NH⁺, Et₄N⁺ or Bu₄N⁺.

13. The process of any one of the preceding claims, wherein the cation scavenger has a structure according to formula Ia: 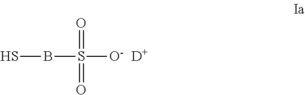
wherein B and D⁺ are as defined in any one of the preceding claims.

14. The process of any one of claims 1-12, wherein the cation scavenger has a structure according to formula Ie, If or Ig: 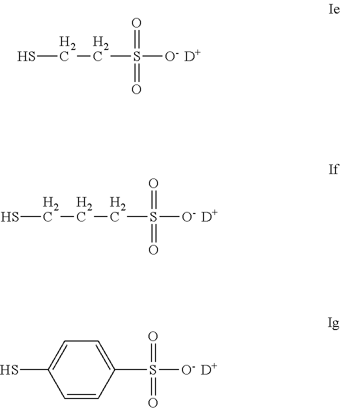
wherein D⁺ is as defined in any one of the preceding claims.

15. The process of any one of the preceding claims, wherein the acid capable of removing the acid-labile protecting group is a haloacetic acid, toluene sulfonic acid, hydrochloric acid, sulfuric acid, phosphoric acid or polyphosphoric acid.

16. The process of any one of the preceding claims, wherein the protected organic compound is a nucleotide, amino acid or sugar.

17. The process of any one of claims 1-15, wherein the protected organic compound is a polymer or an oligomer.

18. The process of claim 17, wherein the protected organic compound is a defined monomer sequence polymer.

19. The process of any one of claims 1 to 15, 17 and 18, wherein the protected organic compound is an oligonucleotide, peptide, peptide nucleic acid or oligosaccharide.

20. The process of any one of the preceding claims, wherein the acid-labile protecting group is selected from the group consisting of dimethoxytrityl (DMT), tert-butyl (tBu), tert-butyl oxycarbonyl (Boc), mono-methoxytriphenyl (Mmtr), triphenyl methyl (Tr), pentamethyl dihydrobenzofuran sulfonyl (Pbf), Tetrahydropyranyl (Thp), tetrahydrofuranyl (Thf), para-methoxybenzyl (Pmb) and 2,4-dimethoxybenzyl.

21. The process of any one of the preceding claims, further comprising the step of isolating the organic compound, once deprotected.

22. The process of claim 21, wherein the organic compound, once deprotected, is isolated by organic solvent nanofiltration.

23. A method for the preparation of a defined monomer sequence polymer by sequential coupling of monomeric units, wherein the method comprises one or more deprotection processes as defined in any one of the preceding claims.

24. The method of claim 23, wherein the defined monomer sequence polymer is an oligonucleotide or a polymer having a polyethylene glycol backbone.

25. A cation scavenger as defined in any one of the preceding claims.
